(19) United States
(12) Patent Application Publication
(10) Pub. No.: US 2022/0401374 A1
(43) Pub. Date: Dec. 22, 2022

(54) COMPOSITIONS AND METHODS FOR CANCER TREATMENT BY ENHANCING ANTITUMOR IMMUNITY USING TANNIC ACID-BASED NANOCAPSULES

(71) Applicant: Purdue Research Foundation, West Lafayette, IN (US)

(72) Inventors: Yoon Yeo, West Lafayette, IN (US); Maie S. Taha, Giza, EG (EG)

(73) Assignee: Purdue Research Foundation, West Lafayette, IN (US)

(21) Appl. No.: 17/641,124

(22) PCT Filed: Sep. 11, 2020
(86) PCT No.: PCT/US20/50284

§ 371 Date: Mar. 8, 2022

Related U.S. Application Data
(60) Provisional application No. 62/899,978, filed on Sep. 13, 2019.

(51) Int. Cl. A61K 9/50; A61K 9/48; A61K 38/07; A61P 35/00

(52) U.S. Cl. CPC A61K 9/5052; A61K 9/4858; A61K 38/07; A61P 35/00

(57) ABSTRACT

The present disclosure generally relates to a composition matter and a method for cancer treatment comprising albumin (alb) coated tannic acid-Fe nanoparticles (NPs) with a therapeutic compound, and one or more diluents, excipients or carriers. In particular, the present invention provides a treatment by enhancing antitumor immunity using tannic acid-based nanoparticles containing a therapeutic cancer treatment that can induce immunogenic cell death (ICD). The method disclosed herein provides a potential solution to the immunotoxicity accompanying the ICD cancer immunotherapy by intratumoral or intravenous administration of a nanocapsule formulation of carfilzomib (CFZ), an ICD-inducing proteasome inhibitor, using interfacial supramolecular assembly of tannic acid (TA) and iron, supplemented with albumin coating for better metabolic stability.

### CROSS REFERENCE TO RELATED APPLICATION

This present patent application relates to and claims the priority under 35 U.S.C. § 119(e) to U.S. Provisional Patent Application Ser. No. 62/899,978 filed on Sep. 13, 2019, the content of which is hereby incorporated by reference in its entirety into the present disclosure.

### GOVERNMENT SUPPORT CLAUSE

This invention was made with government support under a grant CA232419, awarded by the National Institutes of Health. The government has certain rights in the invention.

### TECHNICAL FIELD

The present disclosure generally relates to a composition matter and a method for cancer treatment comprising albumin (alb) coated tannic acid-Fe nanoparticles (NPs) with a therapeutic compound, and one or more diluents, excipients or carriers. In particular, the present invention provides a treatment by enhancing antitumor immunity using tannic acid-based nanoparticles containing a therapeutic cancer treatment that can induce immunogenic cell death (ICD).

### BACKGROUND AND SUMMARY

This section introduces aspects that may help facilitate a better understanding of the disclosure. Accordingly, these statements are to be read in this light and are not to be understood as admissions about what is or is not prior art.

Chemotherapy is one of the main cancer treatments along with surgery, radiation and immunotherapy. Chemotherapeutic drugs interfere with the growth of tumor cells by damaging or blocking the synthesis of nucleic acids, preventing cell division, or inhibiting the homeostatic control of regular cellular functions. Some of the chemotherapeutic drugs have gained increasing interest as immunogenic cell death (ICD) inducers. ICD-inducing chemotherapy kills tumor cells such that it induces the production of tumor-associated antigens and damage-associated molecular patterns (DAMPs), thereby helping the host to develop adaptive immunity to the tumor cells¹. Therefore, ICD inducers can play an important role in cancer immunotherapy as standalone therapeutics to induce specific anti-tumor immune responses² as well as companion drugs to enhance the effect of immune checkpoint blockade therapy³.

Several existing chemotherapeutic drugs are identified to be ICD inducers, which include anthracyclines (doxorubicin, idarubicin, epirubicin), mitoxantrones, oxaliplatin, cyclophosphamide, bortezomib, and paclitaxel^{4,5}. In addition, carfilzomib (CFZ), a second-generation proteasome inhibitor, is considered an emerging ICD inducer⁶, with improved efficacy and safety profiles over the first-generation bortezomib^{7, 8, 9}. CFZ irreversibly inhibits the proteolytic activity of the proteasome⁹, prevents the degradation of misfolded and other key signaling proteins, causing a significant endoplasmic reticulum (ER) stress^{10, 11}, the main mechanism of ICD induction⁶. Therefore, it is expected that CFZ delivered to tumors may produce a spatially defined set of tumor-associated antigens and DAMPs, i.e., in situ tumor vaccines and endogenous immune adjuvants.

Proteasome inhibitors are drugs that block the action of proteasomes, cellular complexes that break down proteins. They are used in the treatment of cancer; and three are approved for use in treating multiple myeloma. Proteasome inhibition may prevent degradation of pro-apoptotic factors such as the p53 protein, permitting the activation of programmed cell death in neoplastic cells dependent upon suppression of pro-apoptotic pathways.

However, a critical challenge in using chemotherapeutic agents to promote cancer immunotherapy is that their antiproliferative effects damage not only tumor cells but also immune cells in the tumor microenvironment, impairing their ability to mount immune responses to dying tumor cells^{12, 13}. Given the paradoxical effect of chemotherapy, it is recognized that optimal regimen is required in order to maximize its therapeutic benefit in the context of cancer immunotherapy¹⁴. In fact, compared to the standard maximum tolerated dose (MTD) regimen, prolonged administration of low doses of chemotherapeutics, called metronomic chemotherapy, has shown reduced immunotoxicity, thereby improving antitumor efficacy¹⁵. Moreover, metronomic dosing of chemotherapeutics can selectively deplete immunosuppressive cell populations, such as myeloid-derived suppressor cells and regulatory T cells, from the tumor microenvironment^{16, 17, 18}. These studies suggest that sustained delivery of ICD inducers to tumors may protect antitumor immune cells and help them to develop effective tumor-specific immune responses¹⁶.

Nanoparticles (NPs) have been widely pursued in the delivery of chemotherapeutics. They have been used to help disperse water-insoluble drugs and/or protect metabolically labile drugs from the hostile physiological environments¹⁹. Moreover, NPs may be designed to control the drug release rate over a prolonged period, facilitating metronomic delivery of chemotherapeutics²⁰. A recent study also reports that NPs can capture tumor neoantigens and DAMPs from dying tumor cells and facilitate their delivery to dendritic cells to activate the antitumor immunity²¹. Therefore, NPs may provide multiple benefits to the delivery of ICD inducers: First, NPs can control the release of ICD inducers to prevent damaging immune cells involved in antitumor immunity. Second, with the relatively large size and drug release control, NPs can retain a drug in tumors longer than the free drug counterpart. Third, NPs can capture the tumor-associated antigens and DAMPs produced by dying tumor cells, improving their exposure to antigen presenting cells.

In the present study, we develop a nanocapsule formulation of CFZ, which can retain the drug in tumors for a prolonged period, control the drug release, and serve as a reservoir of tumor antigens and DAMPs. We hypothesize that a prolonged delivery of CFZ puts tumor cells under extended ER stress to induce ICD^{6, 22, 23, 24}. Sustained release of CFZ at low dose may minimize damages to chemosensitive immune cells recruited to tumors. In addition, as a favored substrate of phagocytes²⁵, nanocapsules capturing tumor antigens and DAMPs may enhance their delivery to dendritic cells (DCs) and subsequent activation of the cells. For this purpose, we encapsulate CFZ in a supramolecular assembly of tannic acid (TA) and iron²⁶ and modified the surface with albumin (CFZ-pTA-alb) to control the drug release. We envision that locally (intratumorally) injected CFZ nanocapsules will activate antitumor immune responses, which can translate to systemic protection against tumors. We evaluate the ability of CFZ nanocapsules to control the drug release and capture soluble proteins released from dying tumor cells. We then compare the effects of CFZ nanocapsules and unformulated CFZ on tumor cells and immune cells in vitro and test if they control tumor growth and develop local and systemic antitumor immunity using mouse models of B16F10 and CT26 tumors.

### BRIEF DESCRIPTION OF THE DRAWINGS

The above and other objects, features, and advantages of the present invention will become more apparent when taken in conjunction with the following description and drawings wherein identical reference numerals have been used, where possible, to designate identical features that are common to the figures, and wherein:

FIGS. 1A-1D show the preparation and characterization of CFZ-pTA-alb nanocapsules: FIG. 1A shows a schematic description of the CFZ-pTA preparation by TA/Fe³⁺ interfacial assembly formation and CFZ-pTA-alb with albumin coating. FIG. 1B shows the TEM images of CFZ-pTA, CFZ-pTA with the CFZ core etched, and CFZ-pTA-alb. Scale bars: 100 nm. FIGS. 1C-1D depict the Z-averages of CFZ-pTA (1C) and CFZ-pTA-alb (1D), incubated in 50% FBS at 37° C. for different times.

FIGS. 2A-2D depict albumin coating as a barrier to CFZ release from CFZ-pTA-alb. FIG. 2A shows the in vitro release kinetics of CFZ-pTA and CFZ-pTA-alb in 10% FBS/RPMI medium. FIG. 2B shows the in vitro stability of CFZ-CD, CFZ-pTA, and CFZ-pTA-alb after incubation at 37° C. in whole blood for 30 min. *: p<0.05, **: p<0.01 and ****: p<0.0001, one-way ANOVA with Tukey's multiple comparisons test. FIG. 2C shows the viability of B16F10 cells exposed to CFZ-DMSO, CFZ-pTA or CFZ-pTA-alb at concentrations equivalent to CFZ 100, 200 or 400 nM for 2 h, followed by incubation in drug-free medium with the total incubation time of 72 h. FIG. 2D shows the viability of B16F10 cells exposed to CFZ-DMSO, CFZ-pTA or CFZ-pTA-alb at 400 nM CFZ-equivalent for 2, 4, 6, 24 or 72 h, followed by incubation in drug-free medium with the total incubation time of 72 h. ***: p<0.001 and ****: p<0.0001, two-way ANOVA with Tukey's multiple comparisons test or Dunnett's multiple comparisons test versus CFZ-DMSO.

FIGS. 3A-3D show comparison of a pulse treatment of bolus dose (bolus-pulse) vs. an extended treatment of low dose (low-extended). Bolus-pulse: Cells were incubated with CFZ-DMSO at different CFZ concentrations for 2.4 h then washed and incubated in drug-free medium for additional 21.6 h making the total incubation time of 24 h. Low-extended: Cells were incubated with at 1/10^{th} CFZ concentrations for 24 h. Cell viability was measured by the MTT assay at the end of 24 h. % cell viability was calculated by normalizing to the viability of control cells treated with the equivalent amount of vehicle for the same period of time. X-axis=CFZ concentration×exposure time (e.g., 2.4=1 nM for 2.4 h followed by incubation in drug-free medium for 21.6 h or 0.1 nM for 24 h). FIG. 3A shows a schematic description of two dosing regimens; FIG. 3B shows viability of splenocytes from C57BL/6 mice; FIG. 3C shows viability of dendritic cells derived from C57BL/6 mouse bone marrow; and FIG. 3D shows viability of B16F10 cells. *: p<0.05, **: p<0.01, ***: p<0.001 and ****: p<0.0001, by two-way ANOVA with Sidak's multiple comparisons test.

FIGS. 4A-4E demonstrate nanocapsules capture DAMPs from dying tumor cells and activate DCs: FIG. 4A shows spectral counts of proteins, analyzed by LC-MS/MS, in the supernatant of B16F10 cells treated with 10 μM CFZ in serum-free medium (B16F10 supernatant), CFZ-pTA-alb (1 mg/mL NP), CFZ-pTA-alb (1 mg/mL NP) incubated in the B16F10 supernatant for 2 h at 37° C. and then rinsed. FIGS. 4B-4C show BMDC activation following coculture with B16F10 cells pretreated with CFZ-DMSO, CFZ-pTA-alb (200 nM CFZ-equivalent) or blank pTA-alb for 24 h, indicated by the expression of (4B) CD40 and (4C) CD86 on CD11c⁺ BMDCs. -: non-activated BMDCs; +: LPS-activated BMDCs. ***: p<0.001 and ****: p<0.0001, one-way ANOVA with Dunnett's multiple comparison's test versus non-activated BMDCs. FIGS. 4D-4E show Phagocytosis of B16F10 cells by BMDCs: B16F10 cells were prelabeled with DiI, treated with CFZ-DMSO, CFZ-pTA-alb (10 μM CFZ-equivalent) or blank pTA-alb, rinsed once and cocultured with BMDCs for (4D) 4 h or (4E) 24 h. BMDCs were identified with APC anti-mouse CD11c antibody and analyzed by flow cytometry. Phagocytosis (%): % of DiI⁺CD11c⁺ cells in total CD11c⁺ cells. ***: p<0.001 and ****: p<0.0001, one-way ANOVA with Dunnett's multiple comparisons test versus non-pretreated B16F10 cells.

FIGS. 5A-5E demonstrate the effects of CFZ-pTA-alb vs. CFZ-CD, administered as a single intratumoral (IT) injection at 1.2 μg CFZ equivalent, on B16F10@C57BL/6 mice. FIG. 5A shows the individual growth curves of tumors treated with PBS (n=4), CFZ-CD (n=5) or CFZ-pTA-alb (n=5). Arrow indicates the time of treatment. FIG. 5B shows the specific growth rate of B16F10 tumor (Δ log V/Δt, V: tumor volume and t: time in days). *: p<0.05 and **: p<0.01, one-way ANOVA with Tukey's multiple comparisons test. FIG. 5C shows the CFZ content in B16F10 tumors and FIG. 5D shows the CFZ concentration in plasma collected at 2 h after IT injection of CFZ-CD (n=4) or CFZ-pTA-alb (n=4) at 1.2 μg CFZ equivalent. *: p<0.05 and **: p<0.01, unpaired two-tailed t-test. FIG. 5E shows the % B16F10 tumor-infiltrating CD8⁺ T cells determined by flow cytometry. *: p<0.05, one-way ANOVA with Tukey's multiple comparisons test.

FIGS. 6A-6D demonstrate the effects of CFZ-pTA-alb vs. CFZ-CD, administered as a single IT injection at 60 μg CFZ equivalent, on the treated (A) and remote (B) tumors in an immunocompetent B16F10@C57BL/6 mouse model. FIG. 6A shows the schedule of B16F10 tumor inoculation in C57BL/6 mice and treatment injection. FIGS. 6B-6C show the antitumor activity in the treated tumor A: FIG. 6B shows the individual growth curves of tumors treated with PBS (n=4), CFZ-CD (n=4) or CFZ-pTA-alb (n=5). Arrow indicates the treatment time. FIG. 6C shows the specific growth rate of treated tumor (Δ log V/Δt). FIG. 6D shows the Days to appearance of tumor B (remote, untreated). *: p<0.05, **: p<0.01 and ***p<0.001, one-way ANOVA with Tukey's multiple comparisons test.

FIGS. 7A-7D demonstrate the effects of CFZ-pTA-alb vs. CFZ-CD, administered as a single IT injection at 60 μg CFZ equivalent, on the treated (A) and remote (B) tumors in an immune compromised B16F10@athymic nude (Foxn1^{nu}) mouse model. FIG. 7A shows the schedule of B16F10 tumor inoculation in athymic nude (Foxn1^{nu}) mice with deficient T cell function and treatment injection. FIG. 7B shows the antitumor activity in the treated tumor A's individual growth curves of tumors treated with PBS (n=4), CFZ-CD (n=4) or CFZ-pTA-alb (n=5). Arrow indicates the treatment time. FIG. 7C shows the antitumor activity in the treated tumor A's specific growth rate of treated tumor (Δ log V/Δt). FIG. 7D shows the days to appearance of tumor B (remote, untreated). **: p<0.01 and ***p<0.001, one-way ANOVA with Tukey's multiple comparisons test.

FIGS. 8A-8C demonstrate the effects of CFZ-pTA-alb vs. CFZ-CD, administered as a single IT injection, on CT26@Balb/c mice. FIG. 8A shows the individual growth curves of tumors treated with PBS (n=3), CFZ-CD (n=3) or CFZ-pTA-alb (n=4) at 1.2 μg CFZ equivalent. Arrows indicate the treatment time. FIG. 8B shows the specific growth rate of CT26 tumor (Δ log V/Δt). FIG. 8C shows the % CD8⁺ T cells of CD45⁺ cells in CT26 tumors or draining lymph nodes of mice receiving a single IT injection of CFZ-CD or CFZ-pTA-alb (n=3 per group) at 60 μg CFZ equivalent, 6 days post treatment. *: p<00.05 and **p<0.01, one-way ANOVA with Tukey's multiple comparisons test.

FIGS. 9A-9D demonstrate the effects of CFZ-pTA-alb vs. CFZ-CD, administered as a single IT injection at 60 μg CFZ equivalent, on the treated (A) and remote (B) tumors in an immunocompetent CT26@Balb/c mouse model. FIG. 9A shows the schedule of CT26 tumor inoculation in Balb/c mice and treatment injection. FIG. 9B shows the antitumor activity in the treated tumor A's individual growth curves of tumors treated with PBS, CFZ-CD or CFZ-pTA-alb (n=5 per group). Arrow indicates the treatment time. FIG. 9C shows the specific growth rate of treated tumor (Δ log V/Δt). FIG. 9D shows the IFN-γ secretion from splenocytes of the mice sacrificed on day 22 post-treatment, in response to AH1 peptide, a CT26 immunodominant MHC class-I restricted antigen. IFN-γ secretion is presented relative to the basal level of IFN-γ in non-challenged splenocytes. *: p<0.05 and **: p<0.01, one-way ANOVA with Tukey's multiple comparisons test.

FIGS. 10A-10G show the effect of CFZ-pTA-alb vs. CFZ-CD, administered by IV injection at 6 mg CFZ-equivalent/kg/dose on two consecutive days, on B16F10@C57BL/6 mice. FIG. 10A depicts individual growth curves of tumors in mice treated with PBS (n=5), CFZ-CD (n=6) or CFZ-pTA-alb (n=9). Arrows indicate the times of treatment. FIG. 10B shows specific tumor growth rate (Δ log V/Δt). ***: p<0.001 and ****: p<0.0001, one-way ANOVA with Tukey's multiple comparisons test. FIGS. 10C-10G show the biodistribution of CFZ in major organs at 48 h after the second dose of CFZ-CD or CFZ-pTA-alb (n=4 per group): Tumor (10C); Liver (10D); Spleen (10E); Lung (10F); Kidney (10G).

### DETAILED DESCRIPTION

While the concepts of the present disclosure are illustrated and described in detail in the figures and the description herein, results in the figures and their description are to be considered as exemplary and not restrictive in character; it being understood that only the illustrative embodiments are shown and described and that all changes and modifications that come within the spirit of the disclosure are desired to be protected.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

The present disclosure generally relates to a composition matter and a method for cancer treatment comprising albumin (alb) coated tannic acid-Fe nanoparticles (NPs) with a therapeutic compound, and one or more diluents, excipients or carriers. In particular, the present invention provides a treatment by enhancing antitumor immunity using tannic acid-based nanoparticles containing a therapeutic cancer treatment that can induce immunogenic cell death (ICD). The method disclosed herein provides a potential solution to the immunotoxicity accompanying the ICD cancer immunotherapy by intratumoral administration of a nanocapsule formulation of carfilzomib (CFZ), an ICD-inducing proteasome inhibitor, using interfacial supramolecular assembly of tannic acid (TA) and iron, supplemented with albumin coating for better metabolic stability.

In some illustrative embodiments, this present disclosure relates to a composition as a cancer treatment comprising albumin (alb) coated tannic acid-Fe (pTA) nanoparticles (NPs) incorporating a therapeutic compound that induces immunogenic cell death (ICD), and one or more diluents, excipients or carriers.

In some illustrative embodiments, this present disclosure relates to a composition as a cancer treatment comprising albumin (alb) coated tannic acid-Fe (pTA) nanoparticles (NPs) incorporating a therapeutic compound that induces immunogenic cell death (ICD), and one or more diluents, excipients or carriers as disclosed herein, wherein said therapeutic compound is an anthracycline or a proteasome inhibitor.

In some illustrative embodiments, this present disclosure relates to a composition as a cancer treatment comprising albumin (alb) coated tannic acid-Fe (pTA) nanoparticles (NPs) incorporating a therapeutic compound that induces immunogenic cell death (ICD), and one or more diluents, excipients or carriers as disclosed herein, wherein said therapeutic compound comprises doxorubicin, idarubicin, epirubicin, mitoxantrones, oxaliplatin, cyclophosphamide, bortezomib, paclitaxel, or carfilzomib (CFZ).

In some illustrative embodiments, this present disclosure relates to a composition as a cancer treatment comprising albumin (alb) coated tannic acid-Fe (pTA) nanoparticles (NPs) incorporating a therapeutic compound that induces immunogenic cell death (ICD), and one or more diluents, excipients or carriers as disclosed herein, wherein said therapeutic compound is carfilzomib (CFZ).

In some illustrative embodiments, this present disclosure relates to a composition as a cancer treatment comprising albumin (alb) coated tannic acid-Fe (pTA) nanoparticles (NPs) incorporating a therapeutic compound that induces immunogenic cell death (ICD), and one or more diluents, excipients or carriers as disclosed herein, wherein said composition is administered locally at the site of a tumor (intratumorally).

In some illustrative embodiments, this present disclosure relates to a composition as a cancer treatment comprising albumin (alb) coated tannic acid-Fe (pTA) nanoparticles (NPs) incorporating a therapeutic compound that induces immunogenic cell death (ICD), and one or more diluents, excipients or carriers as disclosed herein, wherein said composition as a cancer treatment is administered systemically.

In some illustrative embodiments, this present disclosure relates to a composition as a cancer treatment comprising albumin (alb) coated tannic acid-Fe (pTA) nanoparticles (NPs) comprising a therapeutic compound that induces immunogenic cell death (ICD), and one or more diluents, excipients or carriers, wherein said albumin coated tannic acid-Fe nanoparticles with said therapeutic compound are manufactured according to the process of:
a. adding a solution of tannic acid and said therapeutic compound to an aqueous solution of FeCl₃ to afford drug-encapsulated tannic acid-Fe nanoparticles (pTA); and
b. incubating said drug-encapsulated pTA in a solution of albumin for a period of time to afford said nanoparticles.

In some other illustrative embodiments, this present disclosure relates to a composition as a cancer treatment comprising albumin (alb) coated tannic acid-Fe (pTA) nanoparticles (NPs) comprising a therapeutic compound that induces immunogenic cell death (ICD), and one or more diluents, excipients or carriers as disclosed herein, wherein said therapeutic compound that induces immunogenic cell death (ICD) is an anthracycline or a proteasome inhibitor.

In some other illustrative embodiments, this present disclosure relates to a composition as a cancer treatment comprising albumin (alb) coated tannic acid-Fe (pTA) nanoparticles (NPs) comprising a therapeutic compound that induces immunogenic cell death (ICD), and one or more diluents, excipients or carriers as disclosed herein, wherein said therapeutic compound is doxorubicin, idarubicin, epirubicin, mitoxantrones, oxaliplatin, cyclophosphamide, bortezomib, paclitaxel, or carfilzomib (CFZ).

In some other illustrative embodiments, this present disclosure relates to a composition as a cancer treatment comprising albumin (alb) coated tannic acid-Fe (pTA) nanoparticles (NPs) comprising a therapeutic compound that induces immunogenic cell death (ICD), and one or more diluents, excipients or carriers as disclosed herein, wherein said therapeutic compound is carfilzomib (CFZ).

In some other illustrative embodiments, this present disclosure relates to a composition as a cancer treatment comprising albumin (alb) coated tannic acid-Fe (pTA) nanoparticles (NPs) comprising a therapeutic compound that induces immunogenic cell death (ICD), and one or more diluents, excipients or carriers as disclosed herein, wherein said composition is administered locally at the site of a tumor (intratumorally).

In some other illustrative embodiments, this present disclosure relates to a composition as a cancer treatment comprising albumin (alb) coated tannic acid-Fe (pTA) nanoparticles (NPs) comprising a therapeutic compound that induces immunogenic cell death (ICD), and one or more diluents, excipients or carriers as disclosed herein, wherein said composition as a cancer therapy is administered systemically.

In some other illustrative embodiments, this present disclosure relates to a method for treating a patient of cancer comprising the step of administering to a patient in need of relief from said cancer a therapeutically effective amount of a pharmaceutical composition comprising albumin (alb) coated tannic acid-Fe (pTA) nanoparticles (NPs) incorporating a therapeutic compound that induces immunogenic cell death (ICD), and one or more diluents, excipients or carriers, wherein said albumin coated tannic acid-Fe nanoparticles comprising said therapeutic compound are manufactured according to the process of:
a. adding a solution of tannic acid and a therapeutic compound to an aqueous solution of FeCl₃ to afford drug-encapsulated tannic acid-Fe nanoparticles (pTA); and
b. incubating said drug-encapsulated pTA in a solution of albumin for a period of time to afford said nanoparticles.

In some other illustrative embodiments, this present disclosure relates to a method for treating a patient of cancer comprising the step of administering to a patient in need of relief from said cancer a therapeutically effective amount of a pharmaceutical composition comprising albumin (alb) coated tannic acid-Fe (pTA) nanoparticles (NPs) incorporating a therapeutic compound that induces immunogenic cell death (ICD), and one or more diluents, excipients or carriers as disclosed herein, wherein said therapeutic compound that induces immunogenic cell death (ICD) is an anthracycline or a proteasome inhibitor.

In some other illustrative embodiments, this present disclosure relates to a method for treating a patient of cancer comprising the step of administering to a patient in need of relief from said cancer a therapeutically effective amount of a pharmaceutical composition comprising albumin (alb) coated tannic acid-Fe (pTA) nanoparticles (NPs) incorporating a therapeutic compound that induces immunogenic cell death (ICD), and one or more diluents, excipients or carriers as disclosed herein, wherein said therapeutic compound is doxorubicin, idarubicin, epirubicin, mitoxantrones, oxaliplatin, cyclophosphamide, bortezomib, paclitaxel, or carfilzomib (CFZ).

In some other illustrative embodiments, this present disclosure relates to a method for treating a patient of cancer comprising the step of administering to a patient in need of relief from said cancer a therapeutically effective amount of a pharmaceutical composition comprising albumin (alb) coated tannic acid-Fe (pTA) nanoparticles (NPs) incorporating a therapeutic compound that induces immunogenic cell death (ICD), and one or more diluents, excipients or carriers as disclosed herein, wherein said therapeutic compound is carfilzomib (CFZ).

In some other illustrative embodiments, this present disclosure relates to a method for treating a patient of cancer comprising the step of administering to a patient in need of relief from said cancer a therapeutically effective amount of a pharmaceutical composition comprising albumin (alb) coated tannic acid-Fe (pTA) nanoparticles (NPs) incorporating a therapeutic compound that induces immunogenic cell death (ICD), and one or more diluents, excipients or carriers as disclosed herein, wherein said composition is administered locally at the site of a tumor (intratumorally).

In some other illustrative embodiments, this present disclosure relates to a method for treating a patient of cancer comprising the step of administering to a patient in need of relief from said cancer a therapeutically effective amount of a pharmaceutical composition comprising albumin (alb) coated tannic acid-Fe (pTA) nanoparticles (NPs) incorporating a therapeutic compound that induces immunogenic cell death (ICD), and one or more diluents, excipients or carriers as disclosed herein, wherein said composition as a cancer therapy is administered systemically.

In some other illustrative embodiments, this present disclosure relates to a use of a pharmaceutical composition in the preparation of a medicament for treating a cancer, wherein said pharmaceutical composition comprising albumin (alb) coated tannic acid-Fe nanoparticles (NPs) incorporating a therapeutic compound that induces immunogenic cell death (ICD), and one or more diluents, excipients or carriers, wherein said albumin coated tannic acid-Fe nanoparticles comprising said therapeutic compound are manufactured according to the process of:
a. adding a solution of tannic acid and a therapeutic compound to an aqueous solution of FeCl₃ to afford drug-encapsulated tannic acid-Fe nanoparticles (pTA); and
b. incubating said drug-encapsulated pTA in a solution of albumin for a period of time to afford said nanoparticles.

In some other illustrative embodiments, this present disclosure relates to a use of a pharmaceutical composition in the preparation of a medicament for treating a cancer, wherein said pharmaceutical composition comprising albumin (alb) coated tannic acid-Fe nanoparticles (NPs) incorporating a therapeutic compound that induces immunogenic cell death (ICD), and one or more diluents, excipients or carriers as disclosed herein, wherein said therapeutic compound that induces immunogenic cell death (ICD) is an anthracycline or a proteasome inhibitor.

In some other illustrative embodiments, this present disclosure relates to a use of a pharmaceutical composition in the preparation of a medicament for treating a cancer, wherein said pharmaceutical composition comprising albumin (alb) coated tannic acid-Fe nanoparticles (NPs) incorporating a therapeutic compound that induces immunogenic cell death (ICD), and one or more diluents, excipients or carriers as disclosed herein, wherein said therapeutic compound that induces immunogenic cell death (ICD) comprises doxorubicin, idarubicin, epirubicin, mitoxantrones, oxaliplatin, cyclophosphamide, bortezomib, paclitaxel, and carfilzomib (CFZ).

In some other illustrative embodiments, this present disclosure relates to a use of a pharmaceutical composition in the preparation of a medicament for treating a cancer, wherein said pharmaceutical composition comprising albumin (alb) coated tannic acid-Fe nanoparticles (NPs) incorporating a therapeutic compound that induces immunogenic cell death (ICD), and one or more diluents, excipients or carriers as disclosed herein, wherein said therapeutic compound that induces immunogenic cell death (ICD) is carfilzomib (CFZ).

In some other illustrative embodiments, this present disclosure relates to a use of a pharmaceutical composition in the preparation of a medicament for treating a cancer, wherein said pharmaceutical composition comprising albumin (alb) coated tannic acid-Fe nanoparticles (NPs) incorporating a therapeutic compound that induces immunogenic cell death (ICD), and one or more diluents, excipients or carriers as disclosed herein, wherein said therapeutic compound that induces immunogenic cell death (ICD) is administered locally at the site of a tumor (intratumorally).

In some other illustrative embodiments, this present disclosure relates to a use of a pharmaceutical composition in the preparation of a medicament for treating a cancer, wherein said pharmaceutical composition comprising albumin (alb) coated tannic acid-Fe nanoparticles (NPs) incorporating a therapeutic compound that induces immunogenic cell death (ICD), and one or more diluents, excipients or carriers as disclosed herein, wherein said composition as a cancer therapy is administered systemically.

The following examples and detailed embodiments are provided for explanation only, not mean to limit the scope of this disclosure.

Preparation and Characterization of CFZ-pTA and CFZ-pTA-Alb

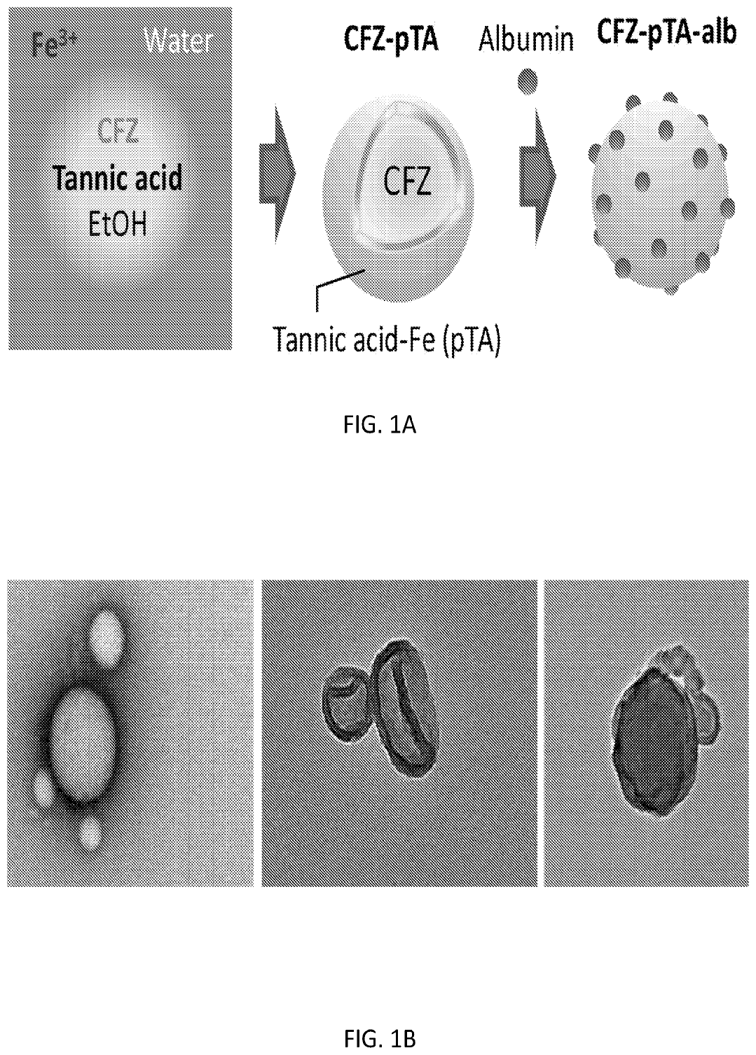

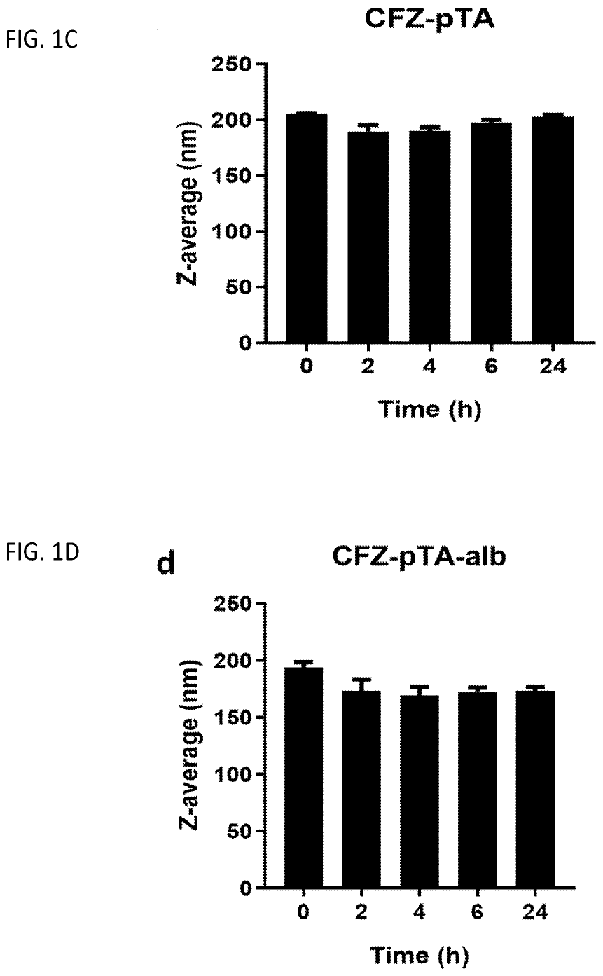

CFZ-pTA nanocapsules were prepared by interfacial assembly of iron-tannic acid complexes²⁶ (FIG. 1A). An ethanolic solution of tannic acid and highly concentrated CFZ was mixed with an aqueous Fe³⁺ solution. Tannic acid and Fe³⁺ formed an instantaneous supramolecular assembly at the interface between ethanol and water, forming spherical nanocapsules containing CFZ, with an average diameter of 100-200 nm (FIG. 1B). The unique dark blue color of the mixture indicated the formation of TA and Fe³⁺ (pTA) complexes. The pTA shell was clearly seen after etching of CFZ core (FIG. 1B). The shell was distinguished from pTA complexes assembled in the absence of CFZ, indicating that the pTA assembly in CFZ-pTA mainly occurred at the interface of ethanol and water. The z-average of CFZ-pTA nanocapsules measured by DLS was 164±1 nm. Their surface charge was measured to be −27±4 mV, reflecting the deprotonated catechol hydroxyl groups of the surface pTA. Interestingly, an ethanolic solution of highly concentrated CFZ (without TA and FeCl₃), mixed with water, also formed nanodroplets with a similar size as CFZ-pTA. The nanodroplets, which have likely resulted from phase separation of supersaturated CFZ, showed positive charge in water, reflecting protonated nitrogens of CFZ. However, CFZ nanodroplets immediately aggregated in phosphate buffer (10 mM, pH 7.4), where the buffer ions neutralized the positive surface charges. In contrast, CFZ-pTA maintained the size in phosphate buffer, indicating the protective effect of pTA assemblies anchored on the surface. CFZ-pTA also showed stable particle size in 50% FBS over 24 h (FIG. 1C) maintaining the spherical structure. This suggests that pTA shell should be stable enough to protect the CFZ-pTA in circulation from disintegration or aggregation. The CFZ content in CFZ-pTA was 51±1 wt % and the TA content 49±7 wt %.

CFZ-pTA was further modified with albumin by 4 h incubation in 2 mg/mL albumin solution. The albumin-coated CFZ-pTA nanocapsules (CFZ-pTA-alb) was similar in size to CFZ-pTA (FIG. 1B) but slightly less charged due to the albumin coverage. The albumin content was estimated to be 15±1 wt % of CFZ-pTA-alb and the CFZ content 41±2 wt %. CFZ-pTA-alb showed colloidal stability in 50% FBS (FIG. 1D), similar to CFZ-pTA.

Albumin Layer of Nanocapsules Attenuates Release and Metabolic Degradation of CFZ

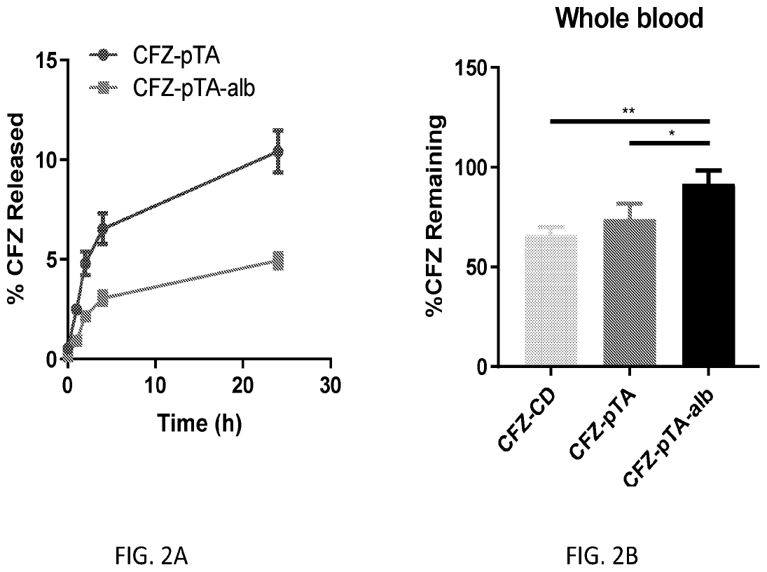

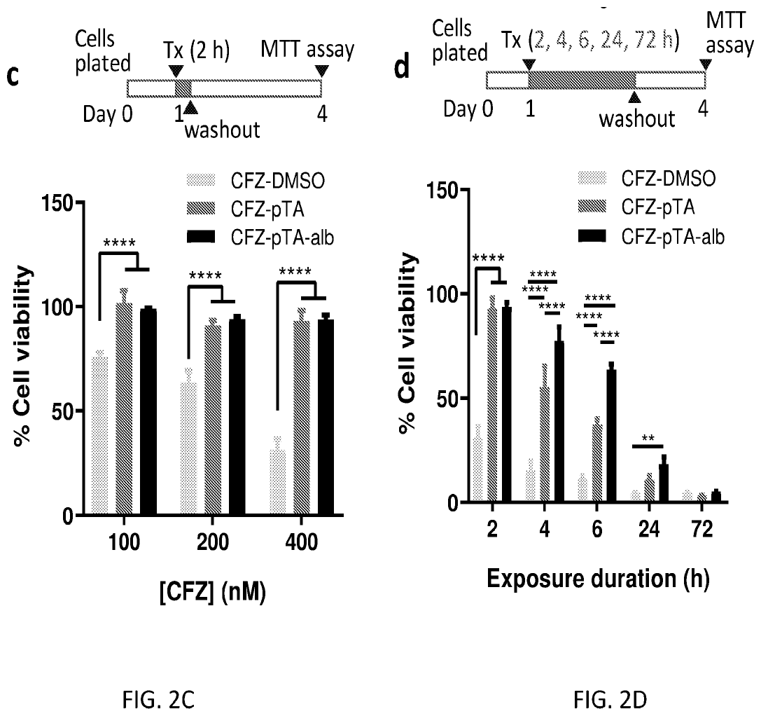

To evaluate the effect of albumin coating on CFZ release control, CFZ-pTA and CFZ-pTA-alb were compared with respect to their CFZ release kinetics in vitro. The particles were first housed in photocrosslinkable PEGDA hydrogel and incubated in 10% FBS-supplemented RPMI 1640 medium, which was sampled at regular intervals to quantify the released CFZ. The hydrogel method was used in lieu of common centrifugation or dialysis methods to avoid the risk of pressurizing and destroying nanocapsules during the repeated centrifugation or excessively diluting the drug to below the detection limit²⁷. The hydrogel confines particles and helps separate them from the medium and does not require a large volume of medium for incubation²⁸. However, the drug release rate measured by this method does not necessarily reflect the actual rate because the hydrogel also functions as a barrier to drug diffusion; thus, the results are only meaningful for rank ordering the two particles. CFZ-pTA-alb showed slower drug release than CFZ-pTA: in 24 h, 5% and 10% of CFZ was released from CFZ-pTA-alb and CFZ-pTA, respectively (FIG. 2A), indicating that albumin coat served as an additional barrier to CFZ release from the particles. CFZ is known for high plasma protein binding (97.6-98.2%)^{29, 30}; therefore, it is conceivable that CFZ escaping CFZ-pTA-alb was temporarily trapped with the surface-bound albumin. A similar result was shown with mitoxantrone-loaded pullulan NPs³¹, where the delayed release of mitoxantrone from albumin-bound NPs was explained by the high affinity of the drug for the surface-bound albumin³².

The stable CFZ encapsulation also enhanced the metabolic stability of CFZ, an epoxyketone peptide, which degrades quickly by peptide cleavage and epoxide ring opening²⁹. Metabolic stability of CFZ in different formulations was compared in vitro after 30 min incubation in whole blood, which contain epoxide hydrolases/peptidases²⁹. With cyclodextrin-solubilized CFZ (CFZ-CD), 66.0±4.0% of total CFZ survived the incubation. CFZ-pTA and CFZ-pTA-alb displayed much improved metabolic stability, with 74.1±7.8% and 91.8±6.6% of CFZ, respectively, remaining under the same conditions (FIG. 2b). This result suggests the protective functions of pTA and albumin. The further protection offered by the albumin coat is consistent with the differential in vitro drug release kinetics (FIG. 2A).

The sustained CFZ release from CFZ-pTA(-alb) also manifested as attenuated cytotoxicity in cancer cell lines. B16F10 (melanoma) cells and HCC-1937 (triple negative breast cancer) were treated with CFZ solubilized in DMSO (CFZ-DMSO), CFZ-pTA, and CFZ-pTA-alb in different concentrations. With 2 h exposure, both CFZ-pTA and CFZ-pTA-alb showed minimal toxicity compared to CFZ-DMSO (FIG. 2C). With extended exposure, nanocapsules gradually caught up with CFZ-DMSO to show comparable cytotoxicities after 72 h exposure, which indicates that CFZ was released from the nanocapsules over time (FIG. 2D). The attenuation of cytotoxicity was more pronounced with CFZ-pTA-alb (FIG. 2D), consistent with its slow drug release per in vitro kinetics data and enhanced stability in whole blood. Blank pTA or pTA-alb showed minimal toxicity in B16F10 cells³³ at concentrations equivalent to CFZ nanocapsules, with 84% (pTA) and 76% (pTA-alb) viability, after maximum exposure (the highest concentration and 72 h exposure). This indicates that cytotoxicity of the nanocapsules is mainly due to CFZ rather than the carriers.

Sustained Release of CFZ Spares Immune Cells

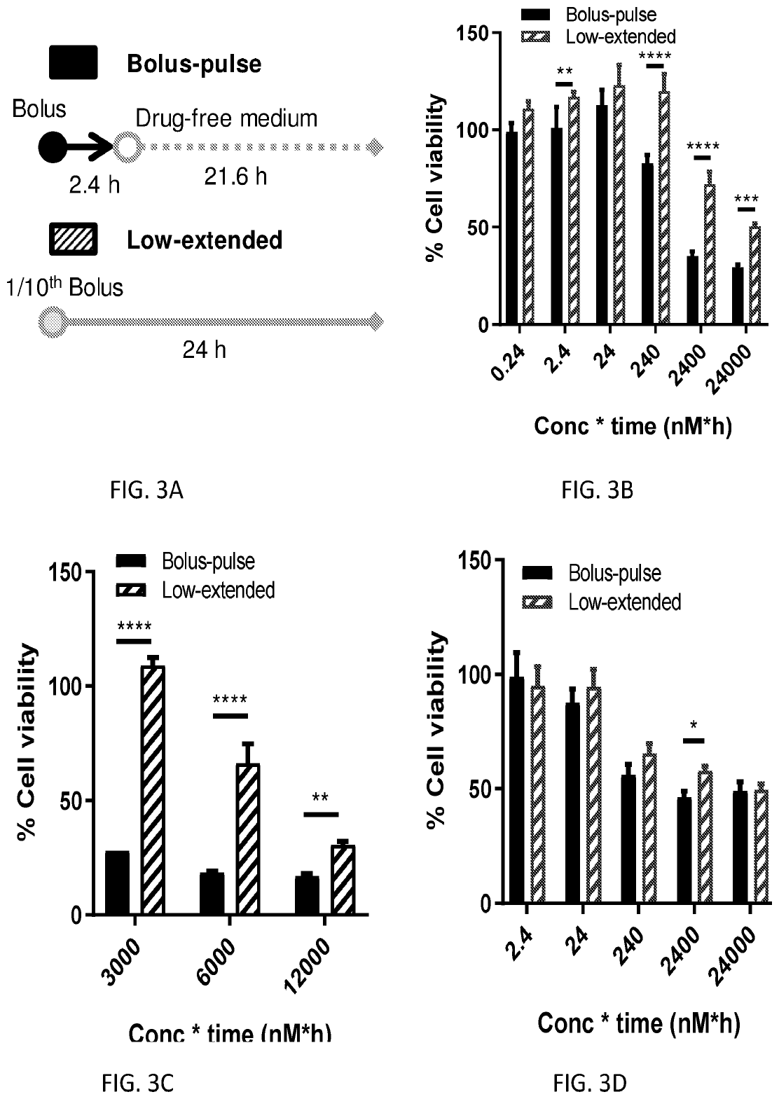

To estimate whether the sustained supply of CFZ is beneficial to sparing immune cells in the tumor microenvironment, we tested the cytotoxic effect of CFZ on splenocytes in two treatment regimens (FIG. 3A): a pulse treatment of bolus dose (bolus-pulse) and an extended treatment of low dose (low-extended), where bolus-pulse indicates incubation of cells in different CFZ concentrations for 2.4 h followed by washout and incubation in drug-free medium for additional 21.6 h making the total incubation time of 24 h, and low-extended indicates incubating cells in 1/10^{th} CFZ concentrations for 24 h. The bolus-pulse regimen represents unformulated CFZ that is immediately available to the tumor upon intratumoral injection but rapidly diffuses to the circulation, whereas the low-extended regimen mimics the sustained release of CFZ from CFZ-pTA-alb over a prolonged period of time. Splenocytes freshly harvested from C57BL/6 mice were used as a surrogate for immune cells, as the spleen is the largest secondary lymphoid organ containing 25% of total lymphocytes³⁴, where B cells and T cells constitute 45-50% and 30-35%, respectively³⁵. Splenocytes were less sensitive to the low-extended regimen than to the bolus-pulse regimen (FIG. 3B). A similar trend was seen with bone marrow-derived dendritic cells (BMDCs) (FIG. 3C). In contrast, B16F10 cells were equally sensitive to the two regimens (FIG. 3D). Taken together these results suggest that the sustained supply of CFZ in the tumor microenvironment, while equally effective against tumor cells as a bolus treatment, be beneficial to maintaining the anti-tumor immunity by protecting tumor infiltrating lymphocytes and DCs.

Nanocapsules Capture DAMPs from Dying Tumor Cells and Enhance DC Uptake of Tumor-Associated Antigens

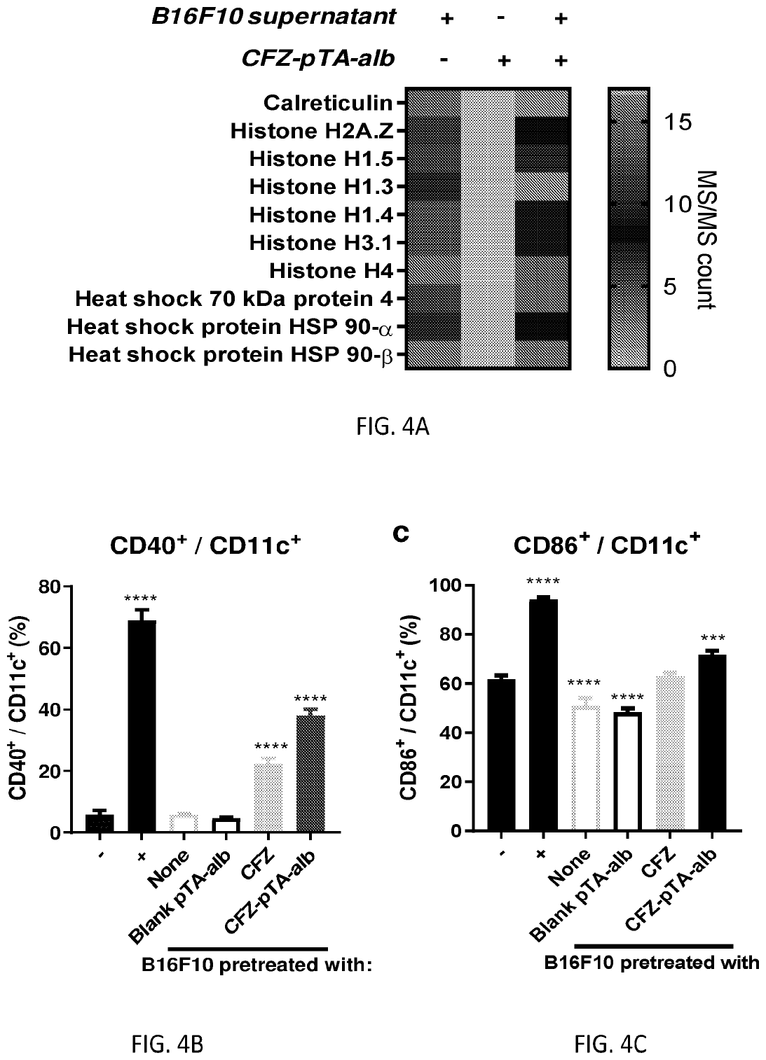

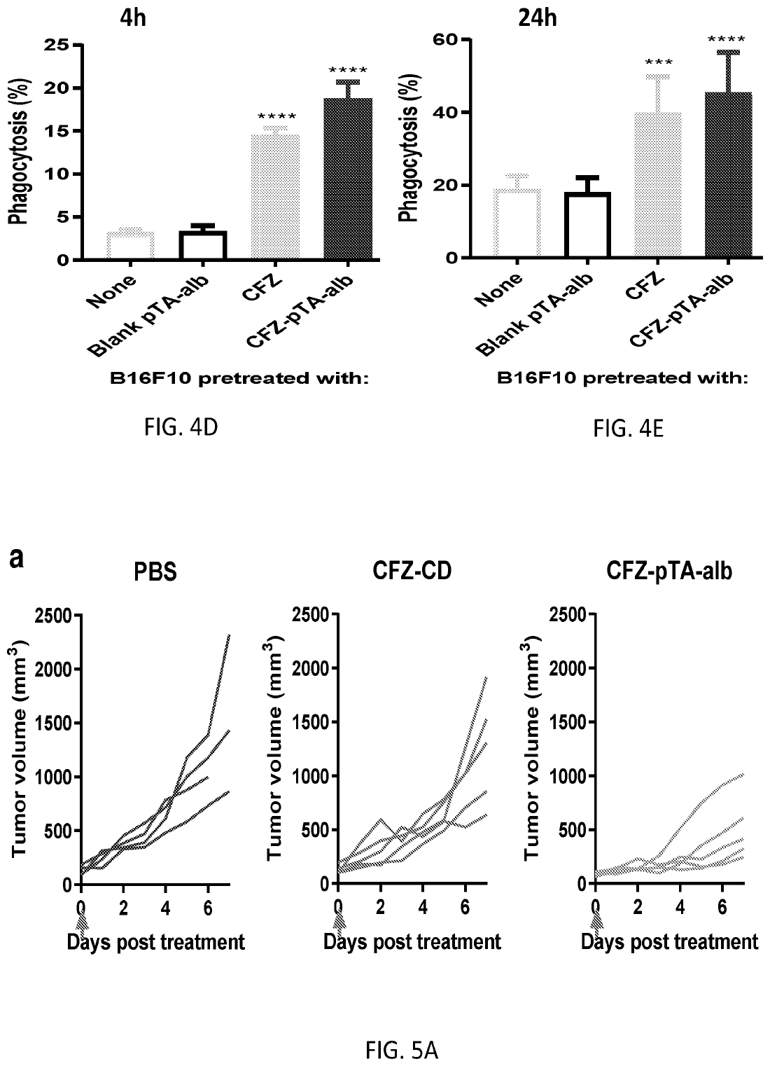

DCs play a central role in development of antitumor immunity. DCs take up tumor antigens, deliver them to draining lymph nodes (DLNs), where they cross-present them to activate T-lymphocytes. A previous study reported that polymeric NPs captured neoantigens and DAMPs of dying tumor cells and delivered them to DCs in DLNs²¹. CFZ-pTA-alb may provide a similar function via the underlying pTA layer, which can interact with proteins via hydrogen bonding and hydrophobic interactions^{36, 37}. To test if CFZ-pTA-alb captures tumor-associated antigens and/or DAMPs, we incubated the nanocapsules in the medium containing soluble proteins released from CFZ-killed B16F10 cells and analyzed the surface-bound proteins. LC-MS/MS analysis showed a large amount of mouse proteins, several of which were identified as DAMPs (Calreticulin, Histone H2A.Z, Histone H1.5, Histone H1.3, Histone H1.4, Histone H3.1, Histone H4, Heat shock 70 kDa protein 4, Heat shock protein HSP 90-α, Heat shock protein HSP 90-β)²¹, captured by CFZ-pTA-alb (FIG. 4A).

We next examined whether the adsorption of DAMPs to nanocapsules increased DC activation. BMDCs were incubated with B16F10 cells pretreated with CFZ, CFZ-pTA-alb or blank-pTA-alb (vehicle) to assess their expression of CD40 and CD86 (DC activation markers) upon their interaction. Untreated or blank pTA-alb-treated (i.e., healthy) B16F10 cells induced no increase in CD40 or CD86 expression in BMDCs. CFZ and CFZ-pTA-alb-treated B16F10 cells increased the expression of the two activation markers, with CFZ-pTA-alb showing a more prominent effect than CFZ (FIGS. 4B and 4C). The DC activation by CFZ-treated B16F10 cells supports the potential of CFZ as an ICD inducer. The relatively high response to CFZ-pTA-alb-treated cells suggest that nanocapsules may have helped to increase local exposure of DAMPs to DCs. Therefore, CFZ-pTA-alb in tumors is expected to not only control the CFZ release but also capture DAMPs from dying cells and deliver them to DCs recruited to the tumor bed or resident in the DLNs to enhance their activation.

Although tumor antigen adsorption was not as evident as DAMP binding, we also examined if the CFZ-pTA-alb helped deliver tumor-associated antigens to DCs by serving as their temporary reservoir. B16F10 cells, stained with DiI (a lipophilic dye) and pretreated with CFZ, CFZ-pTA-alb or blank-pTA-alb (vehicle) for 24 h, were incubated with BMDCs for 4 or 24 h. An excess dose of CFZ (10 μM CFZ equivalent) was used to maximize the effect on B16F10 cells, and the cells were rinsed to remove the extra drug prior to the incubation with BMDCs. BMDCs were identified by anti-CD11c antibody and analyzed by flow cytometry. Cells positive for both DiI and CD11c (DiI⁺CD11c⁺ cells) were considered the BMDCs taking up DiI-stained B16F10 cells and fragments of dying cells. With no apparent toxicity to B16F10 cells, blank-pTA-alb induced no increase in the BMDCs uptake of the treated B16F10 cells (FIGS. 4D and 4E). In contrast, CFZ or CFZ-pTA-alb-pretreated B16F10 cells induced greater extents of DiI⁺CD11c⁺ cells per total CD11c⁺ cells (i.e., BMDCs uptake of DiI⁺ B16F10 cells and their fragments) than the untreated B16F10 cells. The uptake of CFZ-pretreated B16F10 cells by BMDCs was diminished at 4° C., indicating that the uptake was an energy-dependent process, most likely phagocytosis. Of note, CFZ-pTA-alb-treatment induced a greater DiI⁺CD11c⁺ cells fraction than CFZ-treated cells, especially at early incubation (FIGS. 4d and 4e). Confocal microscopy found a consistent trend, where the BMDCs uptake of DiI⁺ cells/fragments increased with CFZ pretreatment, more evidently with CFZ-pTA-alb, according to the Pearson's correlation coefficient of signal colocalization. The membrane staining of BMDCs demonstrated that CFZ-pTA-alb-treated B16F10 cells and fragments were completely internalized by BMDCs. These results suggest that CFZ-pTA-alb facilitate antigen delivery to DCs.

These in vitro results suggest that CFZ-pTA-alb can contribute to the generation of tumor-specific immunity at least in three complementary mechanisms: (i) killing tumor cells to generate tumor-associated antigens by CFZ; (ii) sparing immune cells by sustained release of CFZ; and (iii) serving as a reservoir of DAMPs to enhance the activation and antigen uptake of DCs. On the basis of these results, the effect of intratumorally injected CFZ-pTA-alb on tumor growth and its environment was evaluated by two syngeneic tumor models (B16F10@C57BL/6, CT26@Balb/c).

Local Administration of CFZ-pTA-Alb Provides Better Anti-Tumor Effect than CFZ-CD

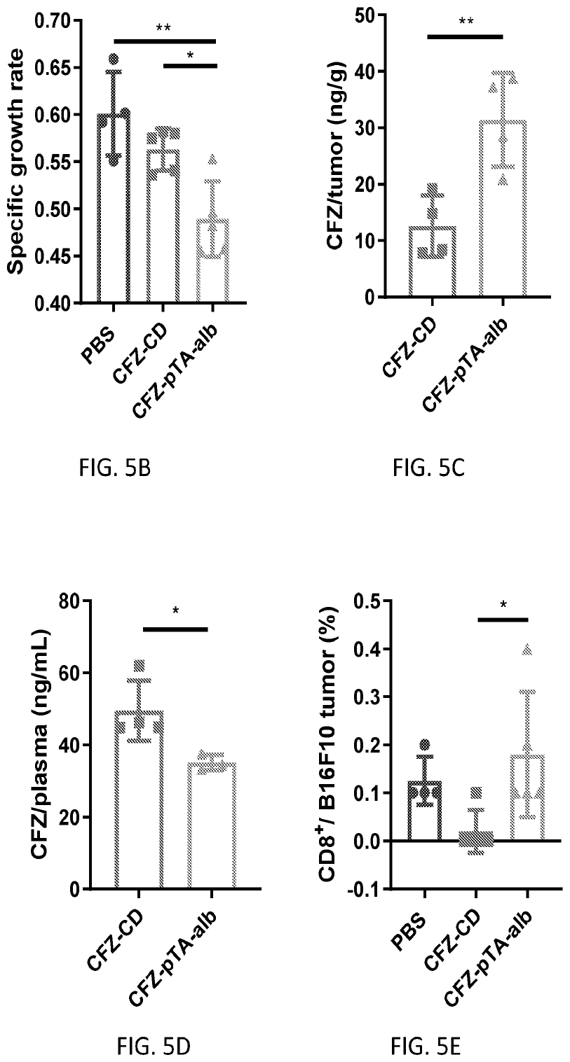

The antitumor effect of CFZ-pTA-alb was evaluated in B16F10 melanoma-bearing C57BL/6 mice and compared with the effect of CFZ-CD (CFZ dissolved in 2-hydroxypropyl-β-cyclodextrin). Subcutaneous B16F10 tumors were injected intratumorally once with PBS, CFZ-CD or CFZ-pTA-alb at a dose equivalent to CFZ 1.2 μg per mouse. With this regimen, CFZ-CD had no significant suppression of tumor growth as compared to PBS control (FIGS. 5A and 5B). However, CFZ-pTA-alb attenuated tumor growth showing significant difference from PBS and CFZ-CD in the specific growth rate (rate of exponential tumor growth calculated for each animal³⁸) (FIGS. 5A and 5B).

The superior effect of CFZ-pTA-lab may be attributed to the prolonged tumor retention of CFZ in the form of nanocapsules, thereby increased local availability of the drug. To test this, another group of identically treated animals were sacrificed 2 h after the treatment, and CFZ in tumors and plasma were quantified. The tumors and plasma were treated with Triton-X 100 to disassemble CFZ-pTA-alb and release CFZ. Therefore, the measured CFZ represents the total detectable amount of CFZ, including both free/released and encapsulated in CFZ-pTA-alb. The amount of CFZ retrieved from CFZ-pTA-alb-treated tumors was 3 times higher than that of CFZ-CD group (FIG. 5C). The CFZ level in plasma showed the opposite trend, with the CFZ-CD group showing a higher level than that of CFZ-pTA-alb (FIG. 5d). This result indicates that CFZ-CD, though freely available from the beginning, was rapidly cleared from the tumor, the scenario simulated by the bolus-pulse regimen in vitro (FIG. 3). On the other hand, the relatively high level of total CFZ in CFZ-pTA-alb-treated tumors suggests that the nanocapsules were better retained in the tumor than CFZ-CD due to the delayed intratumoral transport and lymphatic drainage, making more drug available to tumors. In addition, due to the stability, CFZ-pTA-alb would have provided sustained CFZ exposure to tumor, the scenario represented by the low-extended regimen (FIGS. 3A-3D).

Given the benefit of sustained CFZ release in protecting immune cells predicted in vitro (FIGS. 3b and 3c), we then measured CD8⁺ T cell population in B16F10 tumors harvested at 7 days post-treatment. CFZ-CD-treated tumors apparently showed fewer CD8⁺ T cells than PBS-treated tumors though statistical significance was not reached (FIG. 5e). In contrast, CFZ-pTA-alb-treated tumors had a comparable level of CD8⁺ T cells to the PBS-treated tumors, suggesting difference from CFZ-CD-treated tumors (FIG. 5e). This result is consistent with in vitro prediction and supports that sustained release of CFZ from CFZ-pTA-alb help spare tumor infiltrating cytotoxic lymphocytes.

Local Administration of CFZ-pTA-Alb Helps Develop T-Cell Immunity to Tumors

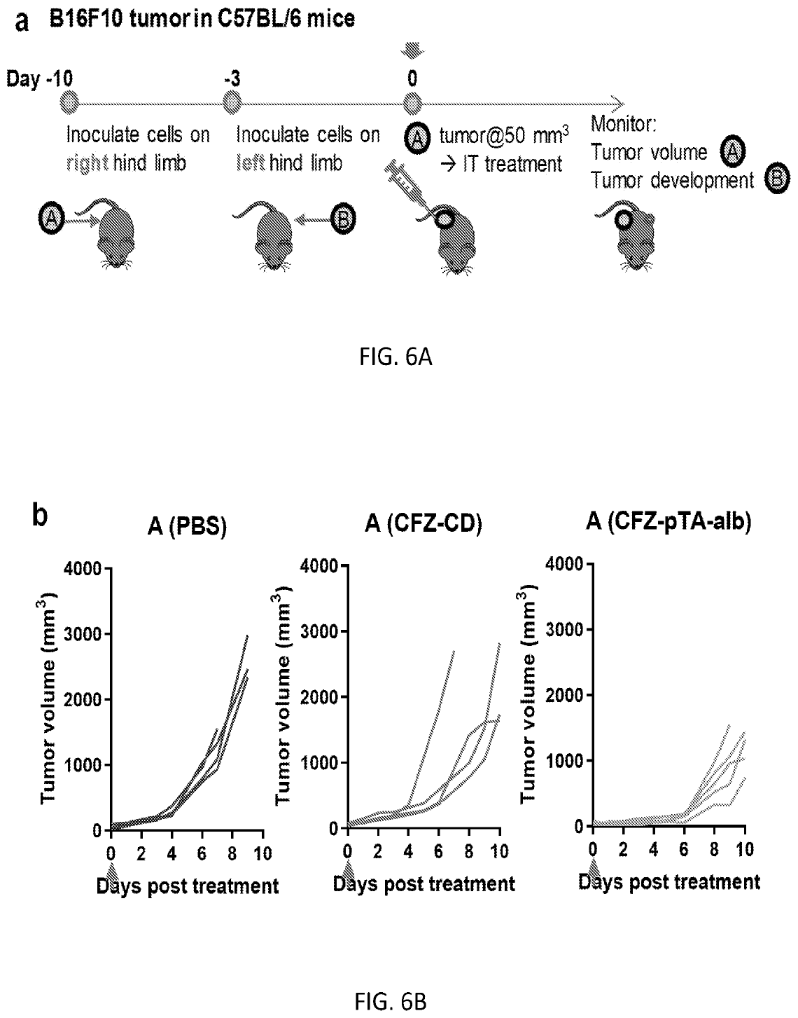

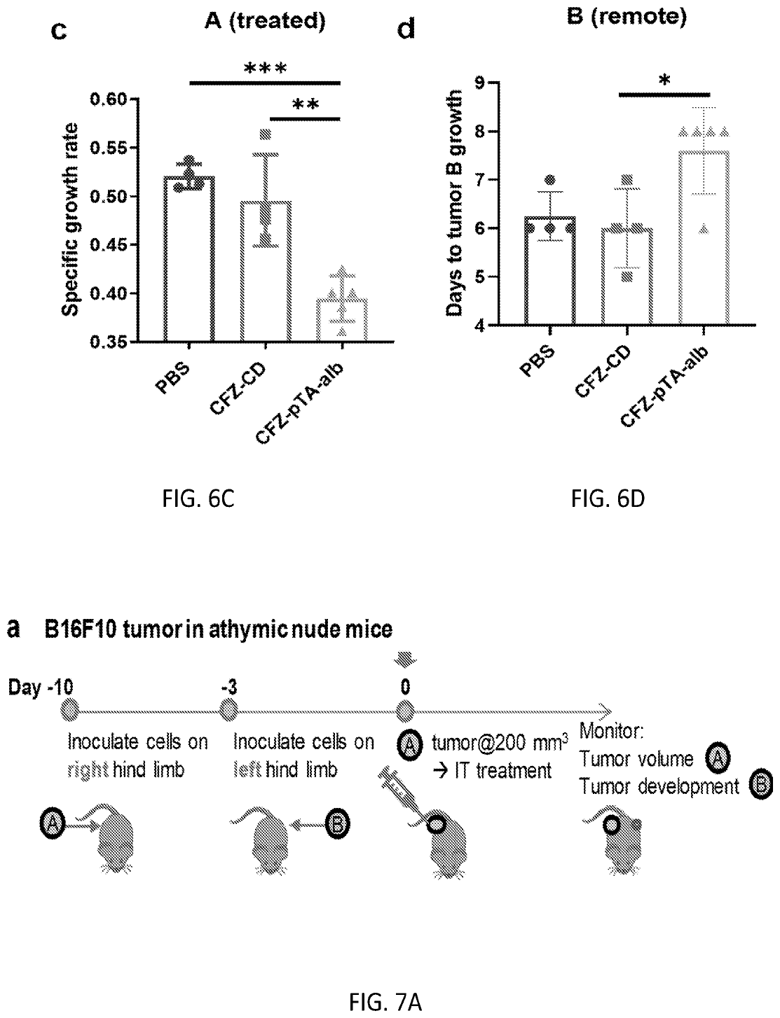

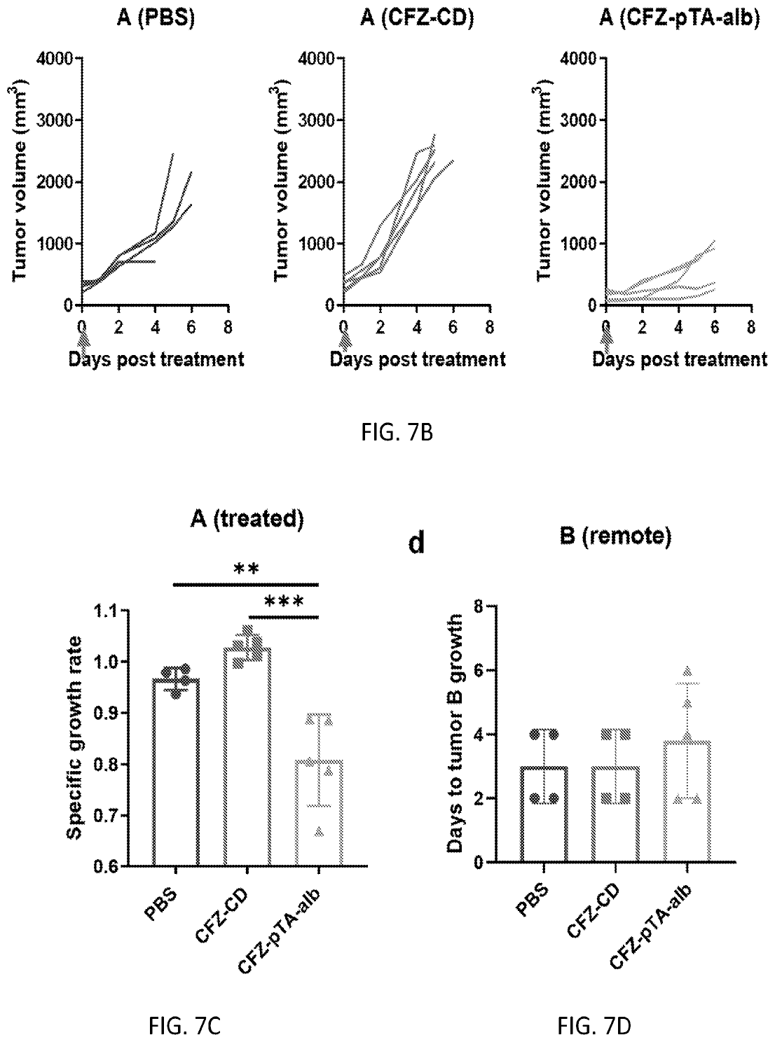

Considering the potential to spare and activate antitumor immune cells in the tumor microenvironment (FIGS. 3b and 3c; FIG. 5e), we asked if the locally administered CFZ-pTA-alb would help develop adaptive immunity to tumors. To test this, we inoculated C57BL/6 mice with B16F10 cells on the flanks of both hind limbs with one-week interval and administered CFZ-pTA-alb (or CFZ-CD, PBS) to the first tumor (tumor A) intratumorally (FIG. 6a). While following up the growth of tumor A, we also monitored the occurrence of tumor on the contralateral side (tumor B), which was left untreated, to observe the ‘abscopal’ effect due to systemic T-cell activation. Consistent with the previous experiment (FIGS. 5a and 5b), CFZ-pTA-alb attenuated the growth of the treated tumor more effectively than the other two treatments (FIGS. 6b and 6c). In addition, CFZ-pTA-alb delayed the emergence of tumor B, whereas CFZ-CD was no different from the PBS control (FIG. 6d). To test whether the effect on tumor B was due to the systemic antitumor immunity or the drug entering the circulation from the injection site, the same test was performed on athymic nude mice with deficient T cell function (FIG. 7a). B16F10-bearing nude mice showed similar responses in the first tumor A, where CFZ-pTA-alb suppressed tumor growth more effectively than the other two treatments (FIGS. 7b and 7c). On the other hand, CFZ-pTA-alb showed no difference from CFZ-CD or PBS in the occurrence of tumor B on the contralateral side (FIG. 7d). This result supports that the effect of local application of CFZ-pTA-alb on the untreated distant tumor shown in the immunocompetent C57BL/6 mice is mediated by the activation of systemic T cell immunity.

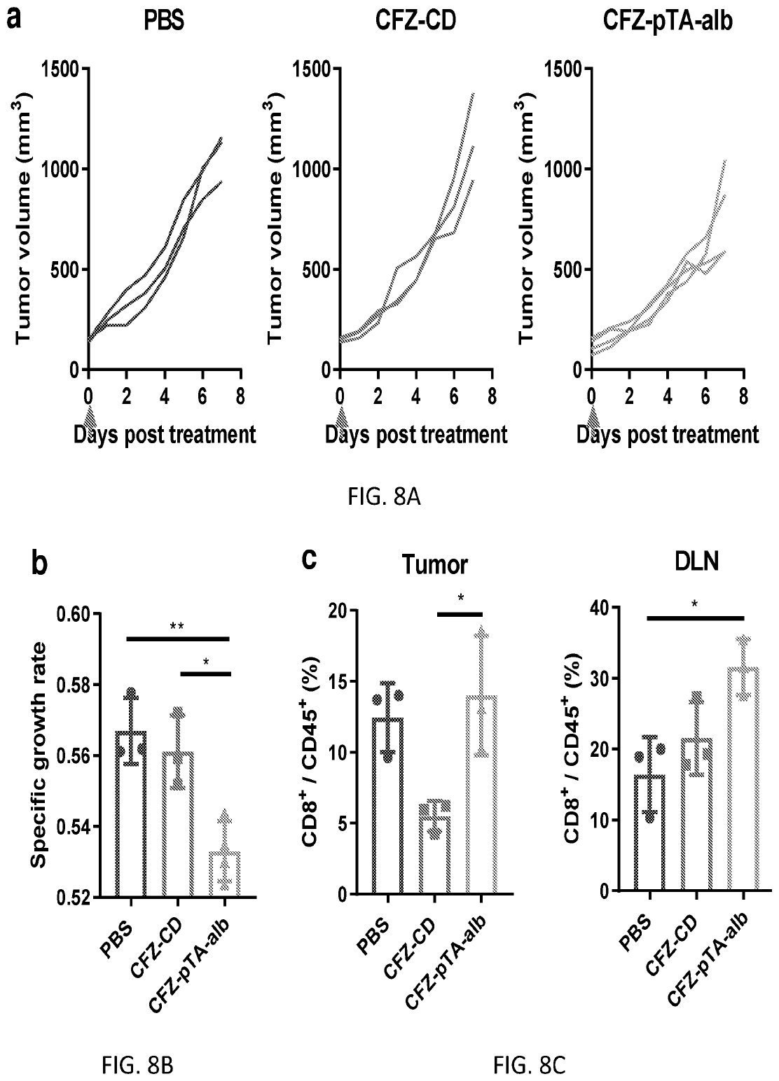

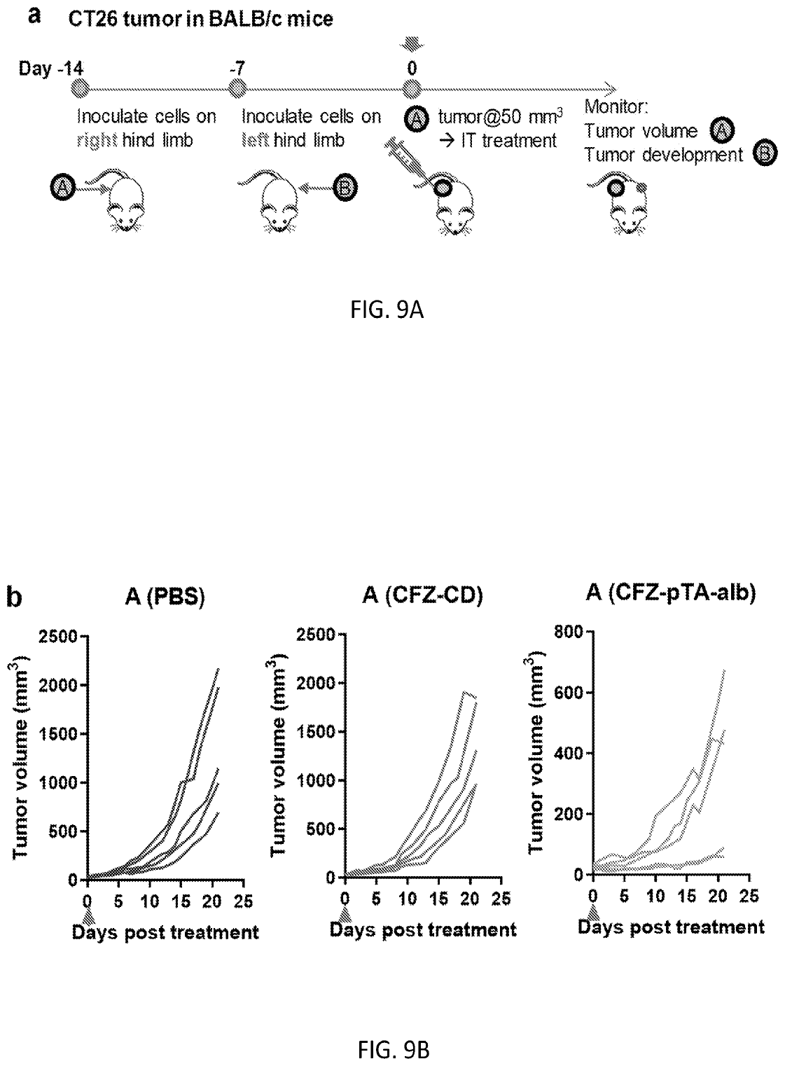

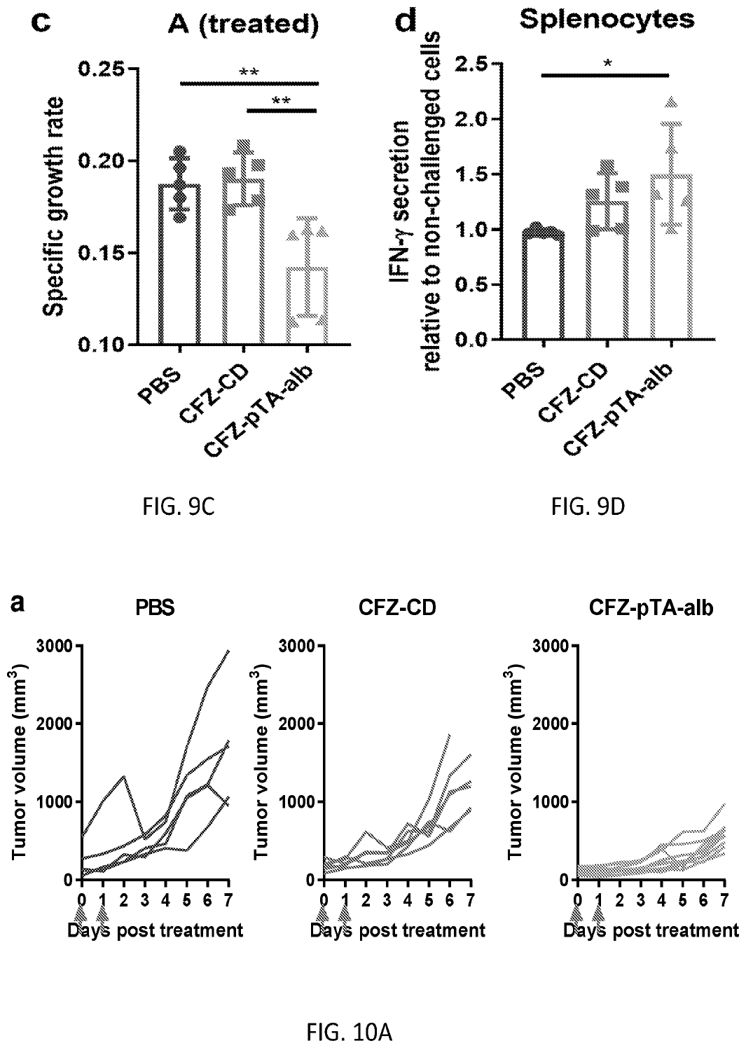

To verify the generation of tumor-specific T-cell immunity, we performed immunophenotyping of the treated tumors and tumor DLNs and tested antigen-specific production of IFN-γ from splenocytes of the treated animals. B16F10 tumors are known to be poorly immunogenic with few tumor-infiltrating cytotoxic T cells³⁹ and, thus, difficult to obtain substantial readouts (FIG. 5e). Therefore, we used the CT26 colon cancer model in syngeneic Balb/c mice, which is relatively more immunogenic due to the high mutational burden⁴⁰ and conducive to monitoring phenotypic changes in tumor immune microenvironment^{39, 41, 42, 43}. CT26 tumors responded to intratumorally-administered treatments in a similar manner as B16F10 tumors, with CFZ-pTA-alb showing better effect than PBS or CFZ-CD (FIGS. 8a and 8b). In addition, the CD8⁺ T cell populations in CT26 tumors and tumor DLNs of the CFZ-pTA-alb group were apparently higher than those of the CFZ-CD group (FIG. 8c), consistent with B16F10 tumors (FIG. 5e) except that the extents were overall higher. The abscopal effect of local treatment was also examined (FIG. 9), but all mice (including PBS-treated ones) did not develop tumor B on the contralateral side, likely due to the concomitant immunity common to immunogenic tumors^{44, 45}, and revealed no difference among the groups. Nevertheless, splenocytes collected from the mice at the conclusion of study (22 days post-treatment) showed a difference in response to AH1 peptide, a CT26 immunodominant MHC class-I restricted antigen⁴⁶. Splenocytes collected from the CFZ-pTA-alb-treated mice showed increase in IFN-γ secretion upon the stimulation with AH1 peptide as compared to those incubated in PBS control (FIG. 9d). In contrast, splenocytes from PBS or CFZ-CD-treated animals did not show significant difference in IFN-γ secretion with and without AH1 peptide stimulation. Of note, the splenocytes of PBS-treated mice produced a relatively high level of IFN-γ irrespective of antigen challenge, likely due to the uncontrolled tumor growth, which induces the accumulation of IFN-γ-producing cells in the spleen via soluble factors^{47, 48}. This result supports that intratumoral administration of CFZ-pTA-alb, but not CFZ-CD, helps develop tumor-specific T-cell immunity, which can affect distant tumors.

Systemic Administration of CFZ-pTA-Alb Provides Better Anti-Tumor Effect than CFZ-CD with Limitation

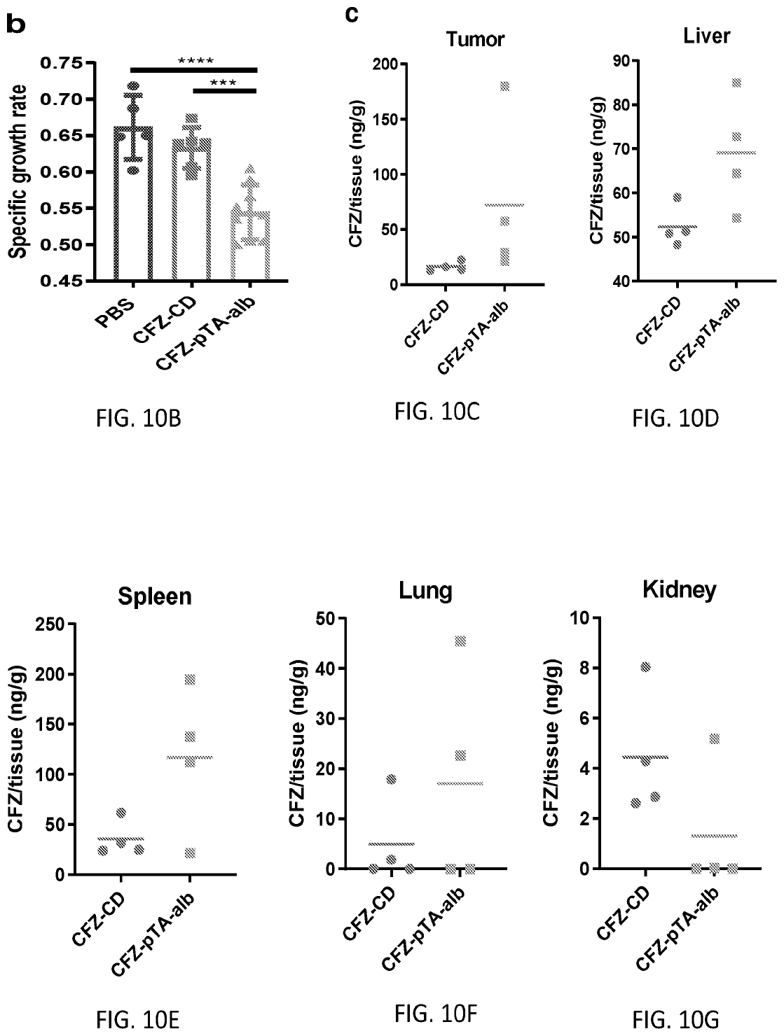

The antitumor effect of CFZ-pTA-alb was also evaluated by systemic administration in C57BL/6 mice bearing B16F10 tumors. Animals were administered with treatments equivalent to CFZ 6 mg/kg/dose⁴⁹ via tail vein on two consecutive days per week (similar to the clinical CFZ regimen for multiple myeloma therapy⁵⁰). With this regimen, CFZ-CD did not differ from the PBS control over 7 days, but CFZ-pTA-alb delayed tumor growth significantly (FIG. 10a). The specific growth rate of the CFZ-pTA-alb-treated group showed statistically significant difference from those of the CFZ-CD and PBS-treated groups (FIG. 10b). This suggests that the systemically administered nanocapsules may have improved CFZ delivery to tumors, as supported by drug distribution profiles at 48 h post-administration (FIGS. 10C-10G). Of note, the detectable amount of CFZ in individual tissues does not include the amount of CFZ that formed the irreversible CFZ-proteasome complex, making it difficult to estimate the total, cumulative amount of CFZ delivered to tumors⁵¹. Nevertheless, CFZ-pTA-alb group showed higher levels of CFZ detected by LC-MS/MS in the tumor, liver, spleen and lungs, as well as lower CFZ in kidneys, typical of NPs (FIGS. 10C-10G).

Despite the initial delay in tumor growth, the animals treated with CFZ-pTA-alb ultimately succumbed to death with additional injections. The mortality did not correlate with the tumor size or body weight loss. We speculate that the metabolic stability offered by CFZ-pTA-alb enhanced the effect of CFZ not only on tumors but also on off-targets such as the MPS organs, which free drug that is rapidly metabolized may not reach but stable nanocapsules may. Blood chemistry measured at 48 h after intravenous (IV) administration of two consecutive doses did not change significantly. However, splenomegaly was observed in healthy mice treated with CFZ-pTA-alb multiple times (eq. to CFZ 6 or 12 mg/kg/dose two consecutive days per week for 2 weeks), which was not seen with blank pTA at an even higher dose. This suggests acute toxicity of CFZ reaching the spleen as NPs.

Discussion In this study we demonstrated the potential to translate local chemotherapy to systemic antitumor immunity via sustained induction of ICD. ICD inducers can generate tumor-associated antigens in situ, thereby activating immune responses against tumors without a prior knowledge of tumor antigens. CFZ, an irreversible proteasome inhibitor, was used as a chemotherapeutic ICD inducer on the basis of its mechanism of action leading to ER stress¹⁰ and the evidence of immunogenic apoptosis⁵². CFZ was better tolerated by immune cells when used at a low dose for an extended period rather than as a high dose bolus (FIGS. 3A-3D), likely due to the overexpression of P-gp efflux proteins on those cells^{53, 54, 55}. As a P-gp substrate⁵⁶, CFZ may be efficiently excluded by the immune cells at a relatively low dose but not at a high dose that saturates the efflux machinery. Therefore, we hypothesize that sustained supply of CFZ will be beneficial to protecting antitumor immune cells in the tumor microenvironment. The sustained release of CFZ was achieved by encapsulation in nanocapsules made of TA/iron interfacial assemblies covered with albumin-CFZ-pTA-alb (FIG. 2A), which serve as effective diffusion barriers to CFZ. The delayed drug release resulted in significant attenuation of in vitro cytotoxicity (FIGS. 2C and 2D). Nevertheless, CFZ-pTA-alb showed consistently greater antitumor effects than CD-solubilized CFZ (CFZ-CD) in vivo by both local and systemic administrations (FIGS. 5-10). Several mechanisms may account for the superior in vivo effects of CFZ-pTA-alb, including the enhanced metabolic stability (FIG. 2B) and prolonged tumor retention of CFZ (FIGS. 5C and 5D). In addition, the effects of local CFZ-pTA-alb administration on remote tumor growth (FIG. 6D), immune cell population in tumor microenvironment (FIGS. 5E, 8C), and the response of splenocytes to a tumor antigen (FIG. 9D) support that CFZ-pTA-alb played positive roles in activating antitumor immunity.

We observed that intratumoral injection of CFZ-pTA-alb to B16F10 tumors grown in immunocompetent C57BL/6 mice delayed the development of the tumor inoculated on the contralateral side, whereas PBS and CFZ-CD had no effect (FIG. 6D). This was not observed in the T-cell deficient athymic nude mice (FIG. 7D), suggesting that the delay in distant tumor growth shown with C57BL/6 mice might be mediated by adaptive immunity. In support of this speculation, the CD8⁺ T cell population in B16F10 tumor remained unaffected in the CFZ-pTA-alb-treated mice, but those treated with CFZ-CD showed few CD8⁺ T cells (FIG. 5E), which is consistent with the resistance of immune cells to the sustained CFZ exposure (FIGS. 3B and 3C). The differential CD8⁺ T cell populations in tumors and DLNs were also observed in another immunocompetent Balb/c mice with CT26 tumors (FIG. 8C). In the CT26 tumor model, there was a general trend that CFZ-pTA-alb-treated mice had greater numbers of CD8⁺ T cells in tumors and DLNs than those treated with PBS, suggesting a potential immunostimulatory effect of the sustained CFZ supply to tumors. Indeed, splenocytes of the Balb/c mice harvested on 22 days after a single intratumoral injection of CFZ-pTA-alb showed increase in the IFN-γ production upon the stimulation with MHC class-I restricted antigen (AH1 peptide), whereas those of PBS-treated mice showed no response to the AH1 peptide (FIG. 9D). The immunostimulatory effect of CFZ-pTA-alb may be attributable, at least partly, to its ability to attract DAMPs based on the adhesive nature of pTA (FIG. 4A), which translates to the increased DC activation and phagocytosis of dying B16F10 cells in vitro (FIGS. 4b-4e). These results collectively support that CFZ-pTA-alb enables the sustained cytotoxic effect to tumor cells with minimal damage to neighboring immune cells and also facilitates the DC activation as a reservoir of DAMPs, which make CFZ-pTA-alb an effective formulation to bridge ICD induction in tumor and promotion of the adaptive antitumor immunity.

This study has focused on the advantage of controlling CFZ release by CFZ-pTA-alb in sparing immune cells in the tumor microenvironment. The literature suggests that the sustained CFZ release in the tumor may have additional benefits. Chemotherapy based on MTDs tend to selectively target chemosensitive cancer cells, leaving behind chemoresistant cell populations that may lead to tumor relapse and emergence of drug resistance⁵⁷. On the contrary, metronomic chemotherapy targets the tumor microenvironment and disengages tumor cells from its support system, resulting in long lasting tumor regression^{16, 18, 57}. In addition, sustained low doses of chemotherapeutics show antiangiogenic effects^{58, 59} and avoid the induction of tumor-initiating cancer stem cells⁵⁷. Bortezomib (the first-generation proteasome inhibitor) has shown to induce endothelial cell apoptosis and downregulate proteins responsible for the angiogenic phenotype in endothelial cells, such as vascular endothelial cell growth factor, interleukin-6, insulin-like growth factor-I, angiopoietin 1 and angiopoietin 2^{60, 61}. The antiangiogenic effect was also reported with oprozomib, an orally active second-generation proteasome inhibitor⁶². As a proteasome inhibitor sharing a similar mechanism of action, CFZ may have an antiangiogenic effect likewise, which would manifest better with sustained release. The contribution of sustained CFZ delivery by CFZ-pTA-alb to antiangiogenesis is worth investigation.

CFZ-pTA-alb was administered as a local (IT) injection in this study. IT injection is clinically feasible for solid tumors that can be located by palpation, imaging or ultrasonography^{63, 64}. Given the specificity and mobility of the activated T cells, the local induction of ICD by IT chemotherapy may be an effective way of inducing systemic antitumor responses with a minimal risk of toxic side effects inherent to systemic chemotherapy^{65, 66, 67}. Nevertheless, it would be ideal if CFZ-pTA-alb can be administered systemically without the need of locating tumors. The IV-injected CFZ-pTA-alb attenuated the growth of B16F10 tumors in C57BL/6 mice (FIGS. 10a and b), consistent with the IT injection (FIGS. 5A and 5B), and showed greater accumulation of CFZ in the tumor than CFZ-CD (FIGS. 10C-10G), which is largely attributable to the enhanced stability of CFZ encapsulated in CFZ-pTA-alb during circulation. However, the animals did not survive the second round of CFZ-pTA-alb by IV injection, likely due to the accumulation of CFZ-pTA-alb in the reticuloendothelial system (RES) organs, which is typical for NPs. Although blood chemistry did not show any signs of abnormality, we suspect that the CFZ-pTA-alb accumulating in those organs with the repeated IV administration may have caused acute toxicity due to the stability of encapsulated CFZ, which would otherwise not have reached them. For systemic application, CFZ-pTA-alb may need to be modified with an additional surface layer to prevent opsonization and reduce the RES accumulation, such as polyethylene glycol. An optimization to maintain the ability to capture DAMPs while preventing the opsonization will be the key to this effort.

To summarize, a nanocapsule formulation of CFZ, CFZ-pTA-alb, was developed for the sustained CFZ delivery and extended ICD induction in solid tumors. Tannic acid/iron assemblies and albumin coating helped control the drug release, enhancing metabolic stability and prolonging tumor retention of CFZ. Moreover, CFZ-pTA-alb helped activate antitumor immunity by selectively sparing immune cells in the tumor microenvironment from CFZ toxicity and serving as a reservoir of immunostimulatory DAMPs released from dying tumor cells. Consequently, CFZ-pTA-alb outperformed CFZ-CD (cyclodextrin-solubilized CFZ) at the equivalent dose in controlling tumor growth and developing local and systemic antitumor immunity in mouse models of B16F10 and CT26 tumors. CFZ-pTA-alb is an effective formulation to translate local ICD induction to systemic antitumor immunity.

Materials and Methods

Carfilzomib (CFZ) was purchased from Shenzhen Chemical Co. LTD. (Shanghai, China). Tannic acid, iron chloride, 2-hydroxypropyl-β-cyclodextrin, human serum albumin (≥96% agarose gel electrophoresis) and Irgacure 2959 were purchased from Sigma-Aldrich (St. Louis, Mo., USA). Polyethyleneglycol dimethylacrylate (PEGDA, MW: 3400 Da) was purchased from Alfa Aesar (Haverhill, Mass., USA). Mouse interferon-gamma (IFN-γ) ELISA kit was purchased from Invitrogen (Eugene, Oreg., USA). GM-CSF was purchased from PeproTech (Rock Hill, N.J., USA). Purified rat anti-mouse CD16/CD32 (Fc block), APC anti-mouse CD11c, FITC anti-mouse CD40, PE anti-mouse CD86, FITC anti-mouse CD4, APC anti-mouse CD8a, and Zombie Violet were purchased from BioLegend (San Diego, Calif., USA). AH1 peptide was purchased from Anaspec (Fremont, Calif., USA). All other materials were of analytical grade and purchased from Thermo Fisher Scientific (Waltham, Mass., USA).

TA/Fe³⁺-Stabilized CFZ Nanocore Preparation and Albumin Coating

CFZ was mixed with TA in 30 μL ethanol at a CFZ to TA molar ratio of 2.4. To this mixture, 10 mL water containing 100 μg FeCl₃ (molar ratio of TA to Fe³⁺: 1.1) was added and mixed briefly with simple agitation to form nanocapsules. The particles were centrifugated at 43,400 rcf for 20 min at 4° C. to remove excess TA and FeCl₃. The formed nanocapsules were called CFZ-pTA, where pTA stands for polymerized tannic acid crosslinked via Fe³⁺. The CFZ-pTA nanocapsules were further incubated in albumin solution (2 mg/mL) in water at CFZ-pTA to albumin weight ratio of 1:2, with mild rotation at room temperature (RT) for 4 h. The particles were centrifuged at 43,400 rcf for 20 min at 4° C. to remove unadsorbed albumin and washed twice by repeated centrifugation. The albumin-coated CFZ-pTA nanocapsules were called CFZ-pTA-alb. As controls, pTA (TA/Fe³⁺ nanoassemblies) and pTA-alb (albumin-coated pTA) were prepared in the same method as above omitting CFZ. Bare CFZ particles (with no pTA) were prepared by adding water to ethanolic solution of CFZ. The particles were suspended in water and stored at 4° C.

Nanocapsule Characterization

Particle Size and Surface Charge

The hydrodynamic diameter (z-ave), polydispersity index (PDI) and zeta potential of particles were measured by dynamic light scattering (DLS) in sodium phosphate buffer (10 mM, pH 7.4) using a Zetasizer Nano-ZS90 (Malvern Instruments, Worcestershire, UK).

Morphology

Particle morphology was examined by transmission electron microscopy (TEM). An aqueous suspension of freshly prepared particles was placed on a carbon coated copper grid (400 mesh), negatively stained with 1% uranyl acetate and allowed to dry in air. The dried grid was examined under an FEI Tecnai T20 transmission electron microscope (Hillsboro, Oreg., USA). To visualize the capsule structure of CFZ-pTA, the core part of the particles was dissolved in PBS containing 0.2% Tween 80 for 6 h. The particles were collected by centrifugation, resuspended in water, put on the grid and processed as above.

CFZ and TA Contents in CFZ-pTA

The CFZ content in CFZ-pTA was determined by C18 reverse phase HPLC (25 cm×4.6 mm, particle size: 5 μm). CFZ-pTA with a premeasured mass was added to an aqueous solution (pH 7.4) containing ethylenediaminetetraacetic acid (EDTA) (100 mM, to remove Fe³⁺) and urea solution (5M, to disrupt hydrogen bonding between TA and CFZ), mixed briefly by vortexing, and mixed with additional acetonitrile (to dissolve CFZ), making the final particle concentration ˜50 μg/mL. The resulting solution was filtered on 0.45 μm syringe filter prior to analysis. As reported previously⁶⁸ with slight modifications, the mobile phase was composed of water and acetonitrile containing 0.05% trifluoroacetic acid and run in an acetonitrile gradient of 40-80% over 22 min at 0.7 mL/min. CFZ was detected with a UV detector at a wavelength of 210 nm.

The TA content in CFZ-pTA was estimated by bicinchoninic acid (BCA) assay (Pierce® BCA assay kit). CFZ-pTA 0.1 mg was suspended in 200 μL of 0.05 N HCl to dissolve TA. The BCA reagent was mixed with the sample in 8:1 v/v ratio at 37° C. for 30 min. The absorbance of the solution was read at 570 nm by a SpectraMax M3 microplate reader (Molecular Device, Sunnyvale, Calif., USA). CFZ solution at an equivalent concentration was treated same way, and the absorbance was subtracted from the reading of CFZ-pTA. The difference was compared to a calibration curve drawn with TA to determine the TA concentration in the sample.

Albumin Content in CFZ-pTA-Alb

The albumin content in CFZ-pTA-alb was determined by sodium dodecyl sulfate polyacrylamide gel electrophoresis (SDS-PAGE). CFZ-pTA-alb with a premeasured mass or standard albumin solutions were prepared in Laemmli buffer containing β-mercaptoethanol and heated at 95° C. for 5 min. The samples were resolved on a 12% SDS-PAGE run at 120 V for 30 min. After staining with QC Colloidal Coomassie Stain and imaged with Azure c300 (Dublin, Calif., USA), the band intensity was analyzed by densitometry (AzureSpot Analysis Software). The albumin content was determined by comparing the band intensities of CFZ-pTA-alb samples and standard albumin solutions.

Size Stability

CFZ-pTA or CFZ-pTA-alb (at a concentration equivalent to CFZ 60 μg/mL, n=3 per group) were incubated in 50% fetal bovine serum (FBS) at 37° C. for 24 h. The suspensions were sampled periodically to measure their particle size by the Zetasizer.

In Vitro CFZ Release Kinetics

CFZ-pTA or CFZ-pTA equivalent to 10 μg of CFZ were suspended in 0.25 mL of 10% PEG dimethylacrylate (PEGDA, 3400 Da) solution in PBS. The suspensions were illuminated under UV (365 nm) for 10 min in the presence of 25 μL of irgacure solution (20% w/v in methanol) to crosslink PEGDA. The crosslinked PEGDA matrix was briefly rinsed with water to remove free particles and immersed in 1 mL of RPMI-1640 medium supplemented with 10% FBS as a release medium (n=3 per group). The matrix was then incubated at 37° C. on an orbital shaker, and the release medium was completely removed for HPLC analysis and replaced with 1 mL of fresh medium at predetermined time points.

Cytotoxicity of CFZ

B16F10 (derived from murine melanoma; ATCC, Manassas, Va., USA) or HCC-1937 (derived from human triple negative breast cancer; ATCC, Manassas, Va., USA) were grown in RPMI 1640 or DMEM medium, respectively, supplemented with 10% FBS and penicillin (100 IU/mL) and streptomycin (100 μg/mL). Cells were seeded in a 96 well plate at a density of 4,000 cells per well. After 24 h incubation, the culture medium was replaced with fresh medium containing CFZ (as a stock solution in DMSO), CFZ-pTA or CFZ-pTA-alb. The cells were subject to either continuous treatment (in drug-containing media at concentrations equivalent to CFZ 10-400 nM for 72 h) or pulse treatment (in drug-containing media at concentrations equivalent to CFZ 50-800 nM for 2-24 h, followed by washout and additional incubation in drug-free medium up to 72 h). Cell viability was estimated by the MTT (3-(4,5-dimethylthiazol-2-yl)-2,5-diphenyltetrazolium bromide) assay. Culture medium was replaced with 100 μL of fresh medium and 15 μL of MTT solution (5 mg/mL), and cells were incubated for 4 h. The stop/solubilizing solution was added to dissolve formazan crystals, and the absorbance was read at the wavelength of 562 nm by the SpectraMax M3 microplate reader. The cell viability was calculated by normalizing the measured absorbance to that of control cells that did not receive treatments.

The cytotoxicity of CFZ was also measured on splenocytes. The spleen was freshly harvested from a male C57BL/6 mouse, cut into pieces, and filtered through 70 μm and 40 μm cell strainers sequentially to obtain a single cell suspension. The cell suspension was incubated with 1 mL ammonium-chloride-potassium (ACK) lysis buffer for 1 min to remove red blood cells. The splenocytes, suspended in RPMI 1640 medium supplemented with 10% FBS, penicillin (100 IU/mL) and streptomycin (100 μg/mL), were seeded at 200,000 cells per well in a 96 well plate and treated with CFZ solutions for 24 h or 2.4 h with additional 21.6 h incubation in drug-free medium. Cell viability was measured as described above.

In Vitro Metabolic Stability of CFZ

Metabolic stability of CFZ in different formulations (CFZ-CD, CFZ dissolved in 10 mM citrate buffer (pH 3.1) with 20% (w/v) 2-hydroxypropyl-β-cyclodextrin)⁶⁹, CFZ-pTA and CFZ-pTA-alb) was measured in whole blood. The blood was collected from C57BL/6 male mice and stored in a heparinized tube on ice. CFZ formulations were added to the cold blood at a final concentration of 67 μM (n=3 per group) and incubated at 37° C. for 30 min. The blood sample was mixed with 200 μL of 5% Triton-X 100 and vortexed for 10 min. To this mixture, 600 μL of acetonitrile was added, bath-sonicated for 10 min, and centrifuged at 4500 rcf for 30 min to remove precipitated proteins. The supernatant was analyzed by HPLC⁶⁸. A calibration standard curve was prepared with CFZ doped in blood at final concentrations of 4-278 μM and treated in the same way as the samples immediately.

Protein-Capturing Ability of CFZ-pTA-Alb

B16F10 cells were treated with 10 μM CFZ solution in serum-free RPMI 1640 medium (supplemented with penicillin 100 IU/mL and streptomycin 100 μg/mL) for 48 h. The cells were centrifuged at 300 rcf for 5 min to separate soluble protein antigens and DAMPs released from dying cells. CFZ-pTA-alb was incubated in the collected supernatant at a NP concentration of 1 mg/mL for 2 h at 37° C. with rotation. The particles were centrifuged at 16,100 rcf for 20 min at 4° C., washed with water twice, resuspended in water at a concentration of 4 mg/mL and analyzed by SDS-PAGE. Protein bands were excised and analyzed by LC-MS/MS according to the method described previously⁷⁰.

CFZ-pTA-Alb—Dendritic Cell Interaction

Bone marrow cells were collected from healthy male C57BL/6 as reported previously⁷¹. Mice at the age of 6-8 weeks were sacrificed using CO₂ asphyxiation. Using a syringe, the cavities of femur bones were flushed with RPMI 1640 medium containing penicillin/streptomycin. The collected bone marrow was pipetted several times and passed through a 40 μm cell strainer to obtain single cell suspension. The cells were collected by centrifugation at 500 rcf for 8 min, treated with ACK buffer, rinsed, and suspended in IMDM medium supplemented with 10% FBS, penicillin/streptomycin, 20 ng/mL GM-CSF, and 10 mM β-mercaptoethanol at a density of 2×10⁶ cells per 10 mL to differentiate into dendritic cells (DCs). Additional medium was supplemented three days later, and the floating and loosely adherent cells were collected by centrifugation on day 6. The DC phenotype was confirmed by CD11c staining. In a 6-well plate, 10⁵ DCs were cocultured for 4 or 24 h with 4×10⁵ DiI-labeled B16F10 cells (denoted as *B16F10 cells), which had been left untreated or treated with blank pTA-alb, CFZ solution or CFZ-pTA-alb (at 10 μM CFZ equivalent) for 24 h and rinsed once. The co-cultured cells were collected, resuspended in staining buffer, incubated with Fc block for 5 min, and stained with APC-labeled anti-mouse CD11c antibody to determine the extent of phagocytic uptake of *B16F10 cells by dendritic cells. Separately, the co-cultured cells (with unlabeled B16F10 cells) were stained with APC-labeled anti-mouse CD11c, FITC-labeled anti-mouse CD40 and PE-labeled anti-mouse CD86 antibodies to determine the activation status of DCs. The stained cells were analyzed by flow cytometry (Accuri C6, BD Biosciences, San Jose, Calif., USA). Fluorescence-minus-one (FMO) controls were used for compensation and gating. The extent of phagocytosis was expressed as the percentage of DiI⁺CD11c⁺ cells per total CD11c⁺ cells. The DC activation was assessed by CD11c⁺CD40⁺ or CD11c⁺CD86⁺ per total CD11c⁺ cells.

Local Administration of CFZ-pTA-Alb

Antitumor Effect in B16F10@C57BL/6 Mice

All animal procedures were approved by Purdue Animal Care and Use Committee, in conformity with the NIH guidelines for the care and use of laboratory animals. Male C57BL/6 mice (5-6 week old, ˜20 g) were purchased from Envigo (Indianapolis, Ind., USA) and acclimatized for 1 week prior to tumor inoculation. One million B16F10 melanoma cells were inoculated subcutaneously to each C57BL/6 mouse in the upper flank of right hind limb. When tumors reached 50-100 mm³ on the average, mice were randomly assigned to 3 groups and received an intratumoral injection of 50 μL of PBS, CFZ-CD or CFZ-pTA-alb (1.2 μg CFZ equivalent). The tumor volume and body weight were measured every day. The tumor length (L) and width (W) were measured using a digital caliper and the volume (V) was calculated as: V=(L×W²)/2). The specific growth rate of the tumor was calculated as Δ log V/Δt (t: time in days)³⁸. The mice were sacrificed at 7 days after the treatment.

B16F10 tumors were collected, cut into small pieces, mechanically disrupted, and filtered through 70 μm and 40 μm cell strainers sequentially to obtain a single cell suspension. The cell suspension was incubated with 3 mL of ACK lysis buffer for 1 min to remove red blood cells. The cells were rinsed with PBS, resuspended in cell staining buffer at a density of 10⁶ cells per 100 μL, incubated with Fc block for 5 min, and stained with PE-labeled anti-mouse CD8a antibody for 1 h at 4° C. The stained cells were analyzed by flow cytometry (Accuri C6, BD Biosciences, San Jose, Calif.).

CFZ Retention in B16F10 Tumor

Fifty microliters of CFZ-CD or CFZ-pTA-alb (1.2 μg CFZ equivalent) were injected intratumorally into 100 mm³ B16F10 tumors inoculated in C57BL/6 mice. Two hours later the mice were sacrificed, blood was collected via cardiac puncture and put in a heparinized tube. Tumors were harvested, washed with PBS and homogenized in cold PBS (pH 7.4) at 100 mg tissue per 400 μL with an Omni Tissue Master 125 homogenizer. A 100 μL of the tumor homogenate was vortex-mixed with 200 μL of 5% Triton-X 100 for 2 min and then mixed with tert-Butyl Methyl Ether (TBME) (1800 μL) containing carfilzomib-d8 (Cayman Chemical, Ann Arbor, Mich., USA) as an internal standard (250 ng/mL) by rotation for 40 min to extract CFZ. The mixture was centrifuged at 4,500 rcf for 15 min, and the organic layer was separated, transferred to a glass vial, and dried in vacuum. The dried sample was dissolved in 100 μL of DMSO, filtered through 0.45 μm syringe filter and analyzed by LC-MS/MS (Agilent triple quadrupole mass spectrometer coupled with the Agilent 1200 Rapid Resolution HPLC, operated in a positive ion mode)⁷². A calibration standard curve was prepared with tumor homogenates doped with CFZ at a concentration of 0.99-500 ng/mL and processed in the same way as the samples. For quantification of CFZ absorbed to the system, 5 μL of plasma was mixed with 40 μL of acetonitrile, containing 250 ng/mL carfilzomib-d8 as an internal standard, and made up to 20 μL with water. The mixture was vortexed for 5 min then centrifuged at 13,000 g for 10 min. The supernatant was analyzed by LC-MS/MS⁷². A calibration standard curve was prepared with plasma doped with CFZ at a concentration of 3.26 to 416.7 ng/mL and processed in the same way as the samples.

Effects on Remote B16F10 Tumors in C57BL/6 Mice or Athymic Nude Mice

C57BL/6 mice were inoculated with 10⁶ B16F10 cells subcutaneously in the upper flank of right hind limb. Seven days later, the mice were inoculated with 3×10⁵ B16F10 cells on the contralateral side. When the first tumor on the right side reached 50 mm³ on the average, mice were randomly assigned to 3 groups. Each mouse received an intratumoral injection of 20 μL of PBS, CFZ-CD or CFZ-pTA-alb (60 μg CFZ equivalent) in the first tumor. The treated tumor and the second inoculation site on the left were monitored every other day. Animals losing >20% body weight loss or with tumors greater than 10% of the body weight were humanely sacrificed prior to the end of the study. The same procedure was repeated with athymic nude (Foxn1^{nu}) mice.

Anti-Tumor Effect and Tumor Immunophenotyping in CT26@Balb/c Mice

10⁵ CT26 mouse colon carcinoma cells (ATCC, Manassas, Va., USA) were inoculated subcutaneously in the mammary fat pad of each female Balb/c mouse. When tumors reached 50-100 mm³ on the average, mice were randomly assigned to 3 groups and received an intratumoral injection of 50 μL of PBS, CFZ-CD or CFZ-pTA-alb (1.2 μg CFZ equivalent). The tumor volume and body weight were measured every day for 7 days. A separate group of CT26 tumor-bearing Balb/c mice (3×10⁵ CT26 cells inoculated subcutaneously to the upper flank of the right hind limb) were treated with PBS, CFZ-CD or CFZ-pTA-alb (60 μg CFZ equivalent) and sacrificed at 6 days post treatment. Tumors were digested in 10 mL of complete RPMI 1640 containing 1 mg/mL collagenase I for 1-2 h, passed through a 70 μm cell strainer to remove large debris, and then pelleted. The samples were treated with 3 mL ACK buffer for 2 min at room temperature, rinsed with 10 volumes of PBS, passed through a 70 μm cell strainer and incubated with Zombie Violet and Fc block at room temperature in dark for 10 min. BV605-labeled anti-mouse CD45 and APC-labeled anti-mouse CD8a antibodies were added to the samples and incubated for 30 min at 4° C. The stained cells were rinsed with PBS and resuspended in 1 mL of 10% neutral buffered formalin for flow cytometry analysis (BD LSRFortessa, San Jose, Calif., USA).

Tumor-Specific Immunity in CT26@Balb/c Mice

Female Balb/c mice were inoculated with 300,000 CT26 cells subcutaneously in the upper flank of right hind limb, followed by 100,000 CT26 cells on the left hind limb 7 days later. When tumors on the right limb grew to ˜50 mm³, the mice were randomly assigned to 3 groups and treated with an intratumoral injection of 20 μL of PBS, CFZ-CD or CFZ-pTA-alb (60 μg CFZ equivalent). The tumor size was monitored for 22 days. At sacrifice, the spleens were collected to evaluate the response to AH1 peptide, a CT26-related peptide antigen. Splenocytes were prepared as described in [00109], plated at a density of 3×10⁵ cells per well in a 96-well plate, and stimulated with 10 μg/mL of AH1 peptide in the presence of 20 ng/mL of GM-CSF. After 72 h incubation, the cells were centrifuged at 300 rcf for 5 min to separate a supernatant. The concentration of interferon-γ (IFN-γ) in each supernatant was measured by ELISA (Invitrogen, Carlsbad, Calif., USA). The IFN-γ production from the AH1 peptide-challenged splenocytes was compared with that of the non-challenged cells collected from the same mouse.

Systemic Administration of CFZ-pTA-Alb

Antitumor Effect in B16F10@C57BL/6 Mice

One million B16F10 melanoma cells were inoculated subcutaneously in the upper flank of the right hind limb of a male C57BL/6 mouse (5-6 week old). When the tumors reached ˜100 mm³, mice were randomly assigned to 3 groups (n=5 for PBS, n=6 for CFZ-CD and n=9 for CFZ-pTA-alb, at a dose equivalent to CFZ 6 mg/kg). One hundred microliters of each treatment were injected via tail vein two consecutive days a week for 2 weeks. The tumor volume and body weight were measured every day.

Blood Chemistry and Tissue Levels of CFZ at 48 h Post-Treatment

Separate groups of C57BL/6 mice with 100 mm³ B16F10 tumors were treated with PBS, CFZ-CD, CFZ-pTA-alb or blank pTA-alb at a dose equivalent to CFZ 6 mg/kg via tail vein injection on two consecutive days. The mice were sacrificed at 48 h after the second injection and bled via terminal cardiac puncture. The serum was separated for analysis of blood chemistry.

The tumor and major organs were harvested, washed with PBS, snap-frozen in liquid nitrogen and kept at −80° C. until analysis. For quantification of CFZ, tissues were homogenized in cold PBS (pH 7.4) at 100 mg tissue per 400 μL with an Omni Tissue Master 125 homogenizer. A 100 μL of each tissue homogenate was vortex-mixed with 200 μL of 5% Triton-X 100 for 2 min and then mixed with TBME (1800 μL) containing carfilzomib-d8 as an internal standard (250 ng/mL) by rotation for 40 min to extract CFZ. The organic layer was separated by centrifugation at 4,500 ref for 15 min, transferred to glass vials and evaporated under vacuum. The dried films were reconstituted in 100 μL acetonitrile and analyzed by LC-MS/MS (Agilent triple quadrupole mass spectrometer coupled with the Agilent 1200 Rapid Resolution HPLC, operated in a positive ion mode)⁷². A calibration standard curve was prepared with tissue homogenates doped with CFZ at a concentration of 0.99-500 ng/mL and processed in the same way as the samples.

Statistical Analysis

Statistical analysis was performed with GraphPad Prism 7 (La Jolla, Calif., USA). All in vitro data were analyzed by unpaired two-way t-test, one-way or two-way ANOVA test to determine the difference of means among groups, followed by the recommended multiple comparisons tests. In vivo data were analyzed by one-way ANOVA, followed by Tukey's multiple comparisons test unless specified otherwise. The comparison of survival curves was conducted with the Log-rank (Mantel-Cox) test. The p-value was indicated for each comparison. A value of p<0.005 was considered statistically significant, and p-values between 0.05 and 0.005 were referred as suggestive, according to the recommendation of the American Statistical Association⁷³.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES

1. Pol J, et al. Trial Watch: Immunogenic cell death inducers for anticancer chemotherapy. OncoImmunology 4, e1008866-e1008866 (2015).
2. Aznar M A, Tinari N, Rullin A J, Sánchez-Paulete A R, Rodriguez-Ruiz M E, Melero I. Intratumoral Delivery of Immunotherapy-Act Locally, Think Globally. The Journal of Immunology 198, 31 (2017).
3. Cui S. Immunogenic Chemotherapy Sensitizes Renal Cancer to Immune Checkpoint Blockade Therapy in Preclinical Models. Med Sci Monit 23, 3360-3366 (2017).
4. Garg A D, Dudek-Peric A M, Romano E, Agostinis P. Immunogenic cell death. Int J Dev Biol 59, 131-140 (2015).
5. Dmitri V K, Abhishek D G, Agnieszka K, Olga K, Patrizia A, Peter V. Immunogenic cell death and DAMPs in cancer therapy. Nature Reviews Cancer 12, 860 (2012).
6. Serrano-del Valle A, Anel A, Naval J, Marzo I. Immunogenic Cell Death and Immunotherapy of Multiple Myeloma. Frontiers in Cell and Developmental Biology 7, (2019).
7. Dick L R, Fleming P E. Building on bortezomib: second-generation proteasome inhibitors as anti-cancer therapy. Drug Discovery Today 15, 243-249 (2010).
8. Palumbo A, et al. Carfilzomib and Dexamethasone Vs Bortezomib and Dexamethasone in Patients with Relapsed Multiple Myeloma: Results of the Phase 3 Study Endeavor (<a href=“pending:yes” 1:ref-type=“CLINTRIALGOV” 1:ref=“NCT01568866”>NCT01568866</a>) According to Age Subgroup. Blood 126, 1844-1844 (2015).
9. Demo S D, et al. Antitumor activity of PR-171, a novel irreversible inhibitor of the proteasome. Cancer Res 67, 6383-6391 (2007).
10. Deshaies R J. Proteotoxic crisis, the ubiquitin-proteasome system, and cancer therapy. BMC Biology 12, 94 (2014).
11. Lamothe B, Wierda W G, Keating M J, Gandhi V. Carfilzomib Triggers Cell Death in Chronic Lymphocytic Leukemia by Inducing Proapoptotic and Endoplasmic Reticulum Stress Responses. Clin Cancer Res 22, 4712-4726 (2016).
12. Verma R, et al. Lymphocyte depletion and repopulation after chemotherapy for primary breast cancer. Breast cancer research: BCR 18, 10 (2016).
13. Litterman A J, et al. Profound impairment of adaptive immune responses by alkylating chemotherapy. J Immunol 190, 6259-6268 (2013).
14. Robertson-Tessi M, El-Kareh A, Goriely A. A model for effects of adaptive immunity on tumor response to chemotherapy and chemoimmunotherapy. Journal of Theoretical Biology 380, 569-584 (2015).
15. Kareva I, Waxman D J, Lakka Klement G. Metronomic chemotherapy: an attractive alternative to maximum tolerated dose therapy that can activate anti-tumor immunity and minimize therapeutic resistance. Cancer letters 358, 100-106 (2015).
16. Chang C L, et al. Dose-dense chemotherapy improves mechanisms of antitumor immune response. Cancer Res 73, 119-127 (2013).
17. Ghiringhelli F, et al. Metronomic cyclophosphamide regimen selectively depletes CD4+CD25+ regulatory T cells and restores T and NK effector functions in end stage cancer patients. Cancer Immunology, Immunotherapy 56, 641-648 (2007).
18. Banissi C, Ghiringhelli F, Chen L, Carpentier A F. Treg depletion with a low-dose metronomic temozolomide regimen in a rat glioma model. Cancer Immunology, Immunotherapy 58, 1627-1634 (2009).
19. Singh R, Lillard J W. Nanoparticle-based targeted drug delivery. Experimental and molecular pathology 86, 215-223 (2009).
20. Jyoti A, Fugit K D, Sethi P, McGarry R C, Anderson B D, Upreti M. An in vitro assessment of liposomal topotecan simulating metronomic chemotherapy in combination with radiation in tumor-endothelial spheroids. Scientific Reports 5, 15236-15236 (2015).
21. Min Y, et al. Antigen-capturing nanoparticles improve the abscopal effect and cancer immunotherapy. Nature Nanotechnology 12, 877-882 (2017).
22. Corazzari M, Gagliardi M, Fimia G M, Piacentini M. Endoplasmic Reticulum Stress, Unfolded Protein Response, and Cancer Cell Fate. Frontiers in oncology 7, 78-78 (2017).
23. Lee A H, Iwakoshi N N, Anderson K C, Glimcher L H. Proteasome inhibitors disrupt the unfolded protein response in myeloma cells. Proc Natl Acad Sci USA 100, 9946-9951 (2003).
24. Urra H, Dufey E, Lisbona F, Rojas-Rivera D, Hetz C. When ER stress reaches a dead end. Biochimica et Biophysica Acta (BBA)—Molecular Cell Research 1833, 3507-3517 (2013).
25. Fang R H, Kroll A V, Zhang L. Nanoparticle-Based Manipulation of Antigen-Presenting Cells for Cancer Immunotherapy. Small (Weinheim an der Bergstrasse, Germany) 11, 5483-5496 (2015).
26. Shen G, Xing R, Zhang N, Chen C, Ma G, Yan X. Interfacial Cohesion and Assembly of Bioadhesive Molecules for Design of Long-Term Stable Hydrophobic Nanodrugs toward Effective Anticancer Therapy. ACS Nano 10, 5720-5729 (2016).
27. Abouelmagd S A, Sun B, Chang A C, Ku Y J, Yeo Y. Release Kinetics Study of Poorly Water-Soluble Drugs from Nanoparticles: Are We Doing It Right? Molecular Pharmaceutics 12, 997-1003 (2015).
28. Sun B, Taha M S, Ramsey B, Torregrosa-Allen S, Elzey B D, Yeo Y. Intraperitoneal chemotherapy of ovarian cancer by hydrogel depot of paclitaxel nanocrystals. Journal of Controlled Release 235, 91-98 (2016).
29. Wang Z, et al. Clinical pharmacokinetics, metabolism, and drug-drug interaction of carfilzomib. Drug metabolism and disposition: the biological fate of chemicals 41, 230-237 (2013).
30. EMA/517040/2016. Group of variations including an extension of indication assessment report. (ed\hat{ }(eds). European Medicines Agency (May 2016).
31. Tao X, et al. Effect of pullulan nanoparticle surface charges on HSA complexation and drug release behavior of HSA-bound nanoparticles. PLoS ONE 7, e49304 (2012).
32. Khan S N, Islam B, Yennamalli R, Sultan A, Subbarao N, Khan A U. Interaction of mitoxantrone with human serum albumin: Spectroscopic and molecular modeling studies. European Journal of Pharmaceutical Sciences 35, 371-382 (2008).
33. Biological evaluation of medical devices—Part 5: Tests for in vitro cytotoxicity. International Organization for Standardization, ISO 10993-10995 (2009).
34. Cesta M F. Normal structure, function, and histology of the spleen. Toxicologic pathology 34, 455-465 (2006).
35. Invitrogen. Cell concentrations in human and mouse samples. (ed\hat{ }(eds).
36. Labieniec M, Gabryelak T. Interactions of tannic acid and its derivatives (ellagic and gallic acid) with calf thymus DNA and bovine serum albumin using spectroscopic method. Journal of photochemistry and photobiology B, Biology 82, 72-78 (2006).
37. Van Buren J P, Robinson W B. Formation of complexes between protein and tannic acid. Journal of Agricultural and Food Chemistry 17, 772-777 (1969).
38. Mehrara E, Forssell-Aronsson E, Ahlman H, Bernhardt P. Specific Growth Rate versus Doubling Time for Quantitative Characterization of Tumor Growth Rate. Cancer Research 67, 3970 (2007).
39. Lechner M G, et al. Immunogenicity of murine solid tumor models as a defining feature of in vivo behavior and response to immunotherapy. Journal of immunotherapy (Hagerstown, Md.: 1997) 36, 477-489 (2013).
40. Castle J C, et al. Immunomic, genomic and transcriptomic characterization of CT26 colorectal carcinoma. BMC Genomics 15, 190 (2014).
41. Casares N, et al. Caspase-dependent immunogenicity of doxorubicin-induced tumor cell death. The Journal of experimental medicine 202, 1691 (2005).
42. Baghdadi M, Chiba S, Yamashina T, Yoshiyama H, Jinushi M. MFG-E8 regulates the immunogenic potential of dendritic cells primed with necrotic cell-mediated inflammatory signals. PLoS ONE 7, e39607-e39607 (2012).
43. Aaes Tania L, et al. Vaccination with Necroptotic Cancer Cells Induces Efficient Anti-tumor Immunity. Cell Reports 15, 274-287 (2016).
44. Lin Y-C, et al. Effector/Memory but Not Naive Regulatory T Cells Are Responsible for the Loss of Concomitant Tumor Immunity. The Journal of Immunology 182, 6095 (2009).
45. Chiarella P, Bruzzo J, Meiss R P, Ruggiero R A. Concomitant tumor resistance. Cancer letters 324, 133-141 (2012).
46. Huang A Y, et al. The immunodominant major histocompatibility complex class I-restricted antigen of a murine colon tumor derives from an endogenous retroviral gene product. Proceedings of the National Academy of Sciences of the United States of America 93, 9730-9735 (1996).
47. Gallina G, et al. Tumors induce a subset of inflammatory monocytes with immunosuppressive activity on CD8+ T cells. The Journal of clinical investigation 116, 2777-2790 (2006).
48. Kano A. Tumor cell secretion of soluble factor(s) for specific immunosuppression. Scientific Reports 5, 8913-8913 (2015).
49. Mehta A, et al. Carfilzomib is an effective anticancer agent in anaplastic thyroid cancer. Endocr Relat Cancer 22, 319-329 (2015).
50. Jakubowiak A J. Evolution of carfilzomib dose and schedule in patients with multiple myeloma: a historical overview. Cancer treatment reviews 40, 781-790 (2014).
51. Park J E, et al. Expanding therapeutic utility of carfilzomib for breast cancer therapy by novel albumin-coated nanocrystal formulation. Journal of Controlled Release 302, 148-159 (2019).
52. Jarauta V, et al. Inhibition of autophagy with chloroquine potentiates carfilzomib-induced apoptosis in myeloma cells in vitro and in vivo. Cancer letters 382, 1-10 (2016).
53. Klimecki W T, Futscher B W, Grogan T_{M}, Dalton W S. P-glycoprotein expression and function in circulating blood cells from normal volunteers. Blood 83, 2451 (1994).
54. Gollapudi S, Gupta S. Anti-P-Glycoprotein Antibody-Induced Apoptosis of Activated Peripheral Blood Lymphocytes: A Possible Role of P-Glycoprotein in Lymphocyte Survival. Journal of Clinical Immunology 21, 420-430 (2001).
55. Cordon-Cardo C, O'Brien J P, Boccia J, Casals D, Bertino J R, Melamed M R. Expression of the multidrug resistance gene product (P-glycoprotein) in human normal and tumor tissues. Journal of Histochemistry & Cytochemistry 38, 1277-1287 (1990).
56. Besse A, et al. Carfilzomib resistance due to ABCB1/MDR1 overexpression is overcome by nelfinavir and lopinavir in multiple myeloma. Leukemia 32, 391-401 (2018).
57. Chan T-S, et al. Metronomic chemotherapy prevents therapy-induced stromal activation and induction of tumor-initiating cells. The Journal of experimental medicine 213, 2967-2988 (2016).
58. Klement G, et al. Continuous low-dose therapy with vinblastine and VEGF receptor-2 antibody induces sustained tumor regression without overt toxicity. J Clin Invest 105, (2000).
59. Browder T, et al. Antiangiogenic Scheduling of Chemotherapy Improves Efficacy against Experimental Drug-resistant Cancer. Cancer Research 60, 1878 (2000).
60. Williams S, Pettaway C, Song R, Papandreou C, Logothetis C, McConkey D J. Differential effects of the proteasome inhibitor bortezomib on apoptosis and angiogenesis in human prostate tumor xenografts. Molecular Cancer Therapeutics 2, 835 (2003).
61. Roccaro A M, et al. Bortezomib Mediates Antiangiogenesis in Multiple Myeloma via Direct and Indirect Effects on Endothelial Cells. Cancer Research 66, 184 (2006).
62. Sanchez E, et al. Anti-angiogenic and anti-multiple myeloma effects of oprozomib (OPZ) alone and in combination with pomalidomide (Pom) and/or dexamethasone (Dex). Leukemia Research 57, 45-54 (2017).
63. Andtbacka R H I, et al. Talimogene Laherparepvec Improves Durable Response Rate in Patients With Advanced Melanoma. Journal of Clinical Oncology 33, 2780-2788 (2015).
64. van Leyen K, Klötzsch C, Harrer J U. Brain Tumor Imaging with Transcranial Sonography: State of the Art and Review of the Literature. Ultraschall in Med 32, 572-581 (2011).
65. Sagiv-Barfi I, et al. Eradication of spontaneous malignancy by local immunotherapy. Science Translational Medicine 10, eaan4488 (2018).
66. Nuhn L, et al. Nanoparticle-Conjugate TLR7/8 Agonist Localized Immunotherapy Provokes Safe Antitumoral Responses. Advanced Materials 30, 1803397 (2018).
67. Burkart C, et al. Improving therapeutic efficacy of IL-12 intratumoral gene electrotransfer through novel plasmid design and modified parameters. Gene Therapy 25, 93-103 (2018).
68. Hayes M E, Noble C O, Francis C. Szoka J. Remote loading of sparingly water-soluble drugs into liposomes. (2014).
69. Park J E, et al. Novel Polymer Micelle and Nanocrystal Formulations for the Proteasome Inhibitor Drug Carfilzomib: Pharmacokinetic and Pharmacodynamic Studies in Human Lung and Breast Cancer Models. The FASEB Journal 31, 822.826-822.826 (2017).
70. Hyun H, et al. Surface modification of polymer nanoparticles with native albumin for enhancing drug delivery to solid tumors. Biomaterials 180, 206-224 (2018).
71. Pang L, Pei Y, Uzunalli G, Hyun H, Lyle L T, Yeo Y. Surface Modification of Polymeric Nanoparticles with M2pep Peptide for Drug Delivery to Tumor-Associated Macrophages. Pharmaceutical Research 36, 65 (2019).
72. Min J S, et al. Quantitative determination of carfilzomib in mouse plasma by liquid chromatography-tandem mass spectrometry and its application to a pharmacokinetic study. Journal of Pharmaceutical and Biomedical Analysis 146, 341-346 (2017).
73. Wasserstein R L, Schirm A L, Lazar N A. Moving to a World Beyond “p<0.05”. The American Statistician 73, 1-19 (2019).

1. A composition as a cancer treatment comprising albumin (alb) coated tannic acid-Fe (pTA) nanoparticles (NPs) incorporating a therapeutic compound that induces immunogenic cell death (ICD), and one or more diluents, excipients or carriers.

2. The composition of claim 1, wherein said therapeutic compound is an anthracycline or a proteasome inhibitor.

3. The composition of claim 1, wherein said therapeutic compound is doxorubicin, idarubicin, epirubicin, mitoxantrones, oxaliplatin, cyclophosphamide, bortezomib, paclitaxel, or carfilzomib (CFZ).

4. The composition of claim 1, wherein said therapeutic compound is carfilzomib (CFZ).

5. The composition of claim 1, wherein said composition is administered locally at the site of a tumor (intratumorally) or systemically.

6. (canceled)

7. The composition as a cancer treatment according to claim 1, wherein said albumin coated tannic acid-Fe nanoparticles with said therapeutic compound are manufactured according to the process of:
a. adding a solution of tannic acid and said therapeutic compound to an aqueous solution of FeCl₃ to afford drug-encapsulated tannic acid-Fe nanoparticles (pTA); and
b. incubating said drug-encapsulated pTA in a solution of albumin for a period of time to afford said nanoparticles.

8. The composition of claim 7, wherein said therapeutic compound that induces immunogenic cell death (ICD) is an anthracycline, doxorubicin, idarubicin, epirubicin, mitoxantrones, oxaliplatin, cyclophosphamide, bortezomib, paclitaxel, and carfilzomib (CFZ), or a proteasome inhibitor.

9. (canceled)

10. (canceled)

11. The composition of claim 7, wherein said composition is administered locally at the site of a tumor (intratumorally) or systemically.

12. (canceled)

13. A method for treating a patient of cancer comprising the step of administering to a patient in need of relief from said cancer a therapeutically effective amount of a pharmaceutical composition comprising albumin (alb) coated tannic acid-Fe (pTA) nanoparticles (NPs) incorporating a therapeutic compound that induces immunogenic cell death (ICD), and one or more diluents, excipients or carriers, wherein said albumin coated tannic acid-Fe nanoparticles comprising said therapeutic compound are manufactured according to the process of:
a. adding a solution of tannic acid and a therapeutic compound to an aqueous solution of FeCl₃ to afford drug-encapsulated tannic acid-Fe nanoparticles (pTA); and
b. incubating said drug-encapsulated pTA in a solution of albumin for a period of time to afford said nanoparticles.

14. The method according to claim 13, wherein said therapeutic compound that induces immunogenic cell death (ICD) is an anthracycline or a proteasome inhibitor.

15. The method according to claim 13, wherein said therapeutic compound comprises doxorubicin, idarubicin, epirubicin, mitoxantrones, oxaliplatin, cyclophosphamide, bortezomib, paclitaxel, and carfilzomib (CFZ).

16. The method according to claim 13, wherein said therapeutic compound is carfilzomib (CFZ).

17. The method according to claim 13, wherein said composition is administered locally at the site of a tumor (intratumorally).

18. The method according to claim 13, wherein said composition as a cancer therapy is administered systemically.

19. Use of a pharmaceutical composition in the preparation of a medicament for treating a cancer, wherein said pharmaceutical composition comprising albumin (alb) coated tannic acid-Fe nanoparticles (NPs) incorporating a therapeutic compound that induces immunogenic cell death (ICD), and one or more diluents, excipients or carriers, wherein said albumin coated tannic acid-Fe nanoparticles comprising said therapeutic compound are manufactured according to the process of:
a. adding a solution of tannic acid and a therapeutic compound to an aqueous solution of FeCl₃ to afford drug-encapsulated tannic acid-Fe nanoparticles (pTA); and
b. incubating said drug-encapsulated pTA in a solution of albumin for a period of time to afford said nanoparticles.

20. The use of a composition in the preparation of a medicament for treating a cancer according to claim 19, wherein said therapeutic compound that induces immunogenic cell death (ICD) is an anthracycline or a proteasome inhibitor.

21. The use of a composition in the preparation of a medicament for treating a cancer according to claim 19, wherein said therapeutic compound that induces immunogenic cell death (ICD) comprises doxorubicin, idarubicin, epirubicin, mitoxantrones, oxaliplatin, cyclophosphamide, bortezomib, paclitaxel, and carfilzomib (CFZ).

22. The use of a composition in the preparation of a medicament for treating a cancer according to claim 19, wherein said therapeutic compound that induces immunogenic cell death (ICD) is carfilzomib (CFZ).

23. The use of a composition in the preparation of a medicament for treating a cancer according to claim 19, wherein said therapeutic compound that induces immunogenic cell death (ICD) is administered locally at the site of a tumor (intratumorally).

24. The use of a composition in the preparation of a medicament for treating a cancer according to claim 19, wherein said composition as a cancer therapy is administered systemically.
